(19) United States
(12) Patent Application Publication
(10) Pub. No.: US 2025/0221917 A1
(43) Pub. Date: Jul. 10, 2025

(54) AQUEOUS DISPERSIONS OF PRE-CROSSLINKED ORGANOPOLYSILOXANES

(71) Applicant: WACKER CHEMIE AG, Munich (DE)

(72) Inventors: Johannes ZIRZLMEIER, Burghausen (DE); Gerhard BEER, Burghausen (DE); Markus GRANDL, Munich (DE)

(73) Assignee: WACKER CHEMIE AG, Munich (DE)

(21) Appl. No.: 18/853,510

(22) PCT Filed: Apr. 6, 2022
(86) PCT No.: PCT/EP2022/059157

§ 371 Date: Oct. 2, 2024

(51) Int. Cl. A61K 8/898; A61Q 5/12; C08G 77/54

(52) U.S. Cl. CPC A61K 8/898; A61Q 5/12; C08G 77/54

(57) ABSTRACT

An aqueous dispersion includes precrosslinked organopolysiloxanes. The precrosslinked organopolysiloxanes contain on average at least one structural unit of the general formula SiRO_{2/2}—Y—SiRO_{2/2} (I), and units of the formula R₂SiO_{2/2} (II), where Y denotes a radical of the formula —R²—[NR³—R⁴]ₓ—NR³—OC—[C(Z¹)(H)]ₖ₁—[C(Z²)(H)]ₖ₂—CO—NR³—[R⁴—NR³]ₐ—R²—R₂SiO_{1/2}—[R₂SiO_{2/2}]_{b}—R₂SiO_{1/2}—R²[NR³—R⁴]ₐ—NR³—OC—[C(Z²)(H)]ₖ₂—[C(Z¹)(H)]ₖ₁—CO—NR³—[R⁴—NR³]ₓ—R².

The invention relates to aqueous dispersions of precrosslinked organopolysiloxanes, to precrosslinked organopolysiloxanes, and to cosmetic compositions comprising aqueous dispersions of precrosslinked organopolysiloxanes. The invention further relates to processes for producing the precrosslinked organopolysiloxanes and aqueous dispersions thereof. The invention further relates to the use of the cosmetic compositions.

Organopolysiloxanes are used in cosmetic compositions, for example in haircare products, on account of their conditioning properties, such as improving softness and smoothness, reducing combing forces, shine properties, improving perceived colors, color protection properties, reducing instances of electrostatic charging, protective properties associated with thermal stress on the hair, or hydrophobization.

An overview of selected organopolysiloxanes for the care of keratin materials such as hair is found in M. D. Berthiaume, Society of Cosmetic Chemists (ed.), monograph, Silicones in Hair Care, 1997 and J. Sejpka, Silicone in Haarpflegeprodukten [Silicones in Haircare Products] in: SÖFW-Journal, volume 118, No. 17, 1992, pp. 1065-1070.

Hair is in everyday life exposed to a multitude of external influences that lead to damage to the hair surface and hence impair the cosmetic properties such as smoothness, softness, shine, and other parameters by comparison with undamaged hair. Damage to the hair surface may be caused for example by chemical or mechanical treatment, by UV radiation or by heat. Accompanying the surface damage to hair is the destruction and partial removal of the lipid layer covering the cuticle, which is responsible for the highly hydrophobic quality of undamaged natural hair (R. A. Lodge, B. Bhusan, Wetting Properties of Human Hair by Means of Dynamic Contact Angle Measurement, Journal of Applied Polymer Science, vol. 102, 5255-5265, 2006, Wiley). Damaged hair is significantly more hydrophilic than undamaged hair, since the effective hair surface following destruction of the superficial lipid layer is a hydrophilic, amino acid-based protein matrix.

Modified siloxanes using oxalamidoester-terminated organopolysiloxanes are known. Those described in WO 2019/114953 A1 are linear copolymers of oxalamidoester-terminated organopolysiloxanes with amino-terminated polyethers. These exhibit significantly increased hydrophilicity compared to exclusively amino-functionalized organopolysiloxanes. However, their linear structure means they have no elastic effect.

U.S. Pat. No. 7,501,184 B2 describes copolymers obtained by reacting linear organopolysiloxanes terminated with oxalamidoester groups with organic diamines. Highly viscous to solid copolymers are obtained, which are used in adhesives, especially as hot-melt adhesives. These highly viscous products are not stably emulsifiable and therefore cannot be used for the treatment of fibrous substrates such as hair. Furthermore, their linear structure means have no elastic effect.

U.S. Pat. No. 7,223,385 B2, U.S. Pat. No. 7,485,289 B2, U.S. Pat. No. 7,220,408 B2, and U.S. Pat. No. 7,504,094 B2 describe cosmetic compositions for treating hair that comprise special amino silicones and also a conditioning agent or a thickener. The amino silicones are dimethylpolysiloxanes with terminal alkoxy/hydroxy groups and with aminoethylaminopropylalkoxysiloxane units or aminoethylaminopropylmethylsiloxane units that are uncrosslinked.

WO 2020/239229 A1 describes aqueous dispersions of precrosslinked organopolysiloxanes, that can be used in cosmetic compositions and that preferably form an elastomeric film after removal of the water. It was found that cosmetic formulations for hair applications comprising the precrosslinked organopolysiloxanes show a particular care effect. Despite the property of forming elastomeric films, only a limited effect on the shaping of hair fibers is observed.

U.S. Pat. No. 5,039,738 A discloses a process for producing modified aminoorganosiloxanes by reacting aminoorganosiloxanes in aqueous emulsions with dialkyl oxalates, dialkyl pyrocarbonates or a mixture thereof. It was found that textile materials treated with the described emulsions exhibit reduced yellowing.

WO 2015/024079 A1 describes cosmetic compositions comprising aminoorganopolysiloxanes, cationic surfactants, and dialkyl esters of dicarboxylic acids of the formula R′—O—CO—R—CO—O—R′, where R′ are C8-C30 radicals.

According to WO 2004/039930 A2, textiles treated with a composition composed of polycarboxylic acids and aminoorganosiloxanes display enhanced resistance to curling and creasing.

A crosslinkable composition composed of aminoorganopolysiloxane and a crosslinker component that is an alkoxysilane or siloxane having at least one carboxylic anhydride group is described in U.S. Pat. No. 5,399,652 A.

The object was to provide precrosslinked organopolysiloxanes, especially aqueous dispersions of precrosslinked organopolysiloxanes, that preferably form an elastomeric film on removal of the water and that can be used in cosmetic compositions. A further object was to provide cosmetic compositions for treating keratin fibers, such as hair, preferably for the cleansing and care thereof, that can be used for shaping and conditioning keratin fibers, such as hair, and in particular provide easier combability thereof.

The object is achieved by the invention.

The invention provides aqueous dispersions, preferably aqueous emulsions, comprising precrosslinked organopolysiloxanes that contain on average at least one structural unit, preferably at least two structural units, more preferably at least three structural units, of the general formula

SiRO_{2/2}—Y—SiRO_{2/2} (I)

and units of the formula

R₂SiO_{2/2} (II)

where
Y denotes a radical of the formula

—R²—[NR³—R⁴]ₓ—NR³—OC—[C(Z¹)(H)]ₖ₁—[C(Z²)(H)]ₖ₂—CO—NR³—[R⁴—NR³]ₐ—R²—R₂SiO_{1/2}-[R₂SiO_{2/2}]_{b}-R₂SiO_{1/2}—R²—[NR³—R⁴]ₐ—NR³—OC—[C(Z²)(H)]ₖ₂—[C(Z¹)(H)]ₖ₁—CO—NR³—[R⁴—NR³]ₓ—R²—
R may be identical or different and denotes a monovalent SiC-bonded hydrocarbon radical that has 1 to 18 carbon atoms and may contain one or more heteroatoms from the group of N, P, S, O, and halogen,
R² may be identical or different and denotes a SiC-bonded, divalent linear or branched hydrocarbon radical having 3 to 18 carbon atoms, preferably an alkylene radical having 3 to 10 carbon atoms,
R³ may be identical or different and denotes a hydrogen atom, an alkyl radical having 1 to 8 carbon atoms or an acyl radical, such as acetyl radical, and preferably is a hydrogen atom,
R⁴ may be identical or different and denotes a divalent hydrocarbon radical having 1 to 6 carbon atoms, preferably an alkylene radical having 1 to 6 carbon atoms,
k1 is 0, 1, 2 or 3, preferably 0,
k2 is 0, 1, 2 or 3, preferably 0,
x is 0, 1, 2, 3 or 4, preferably 0 or 1,
a is 0, 1, 2, 3 or 4, preferably 0 or 1,
b is 0 or an integer from 1 to 500, preferably 20 to 350,
Z¹ denotes —OH, —H or —NHR³, and
Z² denotes —OH, —H or —NHR³.

The invention further provides precrosslinked organopolysiloxanes that contain on average at least one structural unit, preferably at least two structural units, more preferably at least three structural units, of the general formula

SiRO_{2/2}—Y—SiRO_{2/2} (1)
and units of the formula

R₂SiO_{2/2} (II)
where
Y denotes a radical of the formula

—R²—[NR³—R⁴]ₓ—NR³—OC—[C(Z¹)(H)]ₖ₁—[C(Z²)(H)]ₖ₂—CO—NR³—[R⁴—NR³]ₐ—R²—R₂SiO_{1/2}—[R₂SiO_{2/2}]_{b}—R₂SiO_{1/2}—R²—[NR³—R⁴]ₐ—NR³—OC—[C(Z²)(H)]ₖ₂—[C(Z¹)(H)]ₖ₁—CO—NR³—[R⁴—NR³]ₓ—R²—
and
R, R², R³, R⁴, k1, k2, x, a, b, Z¹, and Z² are as defined above.

The invention further provides a process for producing aqueous dispersions of precrosslinked organopolysiloxanes, wherein aqueous dispersions, preferably aqueous emulsions, comprising aminoorganopolysiloxanes (1) of the formula

(R¹O)_{d}AₑR_{3-d-e}SiO(SiARO)ₚ(SiR₂O)_{q}SiR_{3-d-e}Aₑ(OR¹)_{d} (IV)
where
A is an amino radical of the general formula

—R²—[NR³—R⁴—]ₓNR³₂
R may be identical or different and denotes a monovalent SiC-bonded hydrocarbon radical that has 1 to 18 carbon atoms and may contain one or more heteroatoms from the group of N, P, S, O, and halogen,
R¹ may be identical or different and denotes a hydrogen atom or an alkyl radical that has 1 to 18 carbon atoms and may be interrupted by one or more separate oxygen atoms,
R² may be identical or different and denotes a SiC-bonded, divalent linear or branched hydrocarbon radical having 3 to 18 carbon atoms, preferably an alkylene radical having 3 to 10 carbon atoms,
R³ may be identical or different and denotes a hydrogen atom, an alkyl radical having 1 to 8 carbon atoms or an acyl radical, such as acetyl radical, and preferably is a hydrogen atom,
R⁴ may be identical or different and denotes a divalent hydrocarbon radical having 1 to 6 carbon atoms, preferably an alkylene radical having 1 to 6 carbon atoms,
d is 0 or 1,
e is 0 or 1,
p is an integer that is at least 1, preferably at least 2, more preferably at least 3, and at most 1000, preferably at most 10, and
q is 0 or an integer from 1 to 2000, preferably 50 to 1000,
x is 0, 1, 2, 3 or 4, preferably 0 or 1,
with reactive esters (2) of the formula

R⁵—O₂C—[C(Z¹)(H)]ₖ₁—[C(Z²)(H)]ₖ₂—CO—NR³—[R⁴—NR³]ₐ—R²—R₂SiO_{1/2}—[R₂SiO_{2/2}]_{b}—R₂SiO_{1/2}—R²—[NR³—R⁴]ₐ—NR³—OC—[C(Z²)(H)]ₖ₂—[C(Z¹)(H)]ₖ₁—CO₂—R⁵ (V)
where R, R², R³, and R⁴ are as defined above,
R⁵ may be identical or different and denotes an O-bonded, saturated or unsaturated, linear or branched, monovalent hydrocarbon radical that has 1-20 carbon atoms per radical and may contain one or more heteroatoms from the group of N, P, S, O and halogen,
k1 is 0, 1, 2 or 3, preferably 0,
k2 is 0, 1, 2 or 3, preferably 0,
a is 0, 1, 2, 3 or 4, preferably 0 or 1,
b is 0 or an integer from 1 to 500, preferably 20 to 350,
Z¹ denotes —OH, —H or —NHR³, and
Z² denotes —OH, —H or —NHR³.

The invention further provides a process for producing precrosslinked
organopolysiloxanes, characterized in that
aminoorganopolysiloxanes (1) of the formula

(R¹O)_{d}AₑR_{3-d-e}SiO(SiARO)ₚ(SiR₂O)_{q}SiR_{3-d-e}Aₑ(OR¹)_{d} (IV)
are reacted with reactive esters (2) of the formula

R⁵—O₂C—[C(Z¹)(H)]ₖ₁—[C(Z²)(H)]ₖ₂—CO—NR³—[R⁴—NR³]ₐ—R²—R₂SiO_{1/2}—[R₂SiO_{2/2}]_{b}—R₂SiO_{1/2}—R²—[NR³—R⁴]ₐ—NR³—OC—[C(Z²)(H)]ₖ₂—[C(Z¹)(H)]ₖ₁—CO₂—R⁵ (V)
where A, R, R¹, R², R³, R⁴, R⁵, d, e, p, q, k1, k2, x, a, b, Z¹, and Z² are as defined above,
and the resulting precrosslinked organopolysiloxanes are optionally subsequently emulsified in water.

In the process of the invention, aminoorganopolysiloxanes (1) of the formula (IV) that are used are preferably those in which e is 0, and which therefore contain only pendant amino radicals A and in the reaction with the reactive esters (2) form bridges in accordance with structural units of the formula (I).

When e is 0, the precrosslinked organopolysiloxanes of the invention contain not only structural units of the formula (I) and siloxane units of the formula (II) but also, preferably siloxane units of the formula

R_{3-d}(OR¹)_{d}SiO_{1/2} (III)
where R, R¹, and d are as defined above.

It is, however, also possible to use aminoorganopolysiloxanes (1) of the formula (IV) that also contain terminal amino radicals A, in which e is thus 1.

It is also possible to use mixtures of aminoorganopolysiloxanes (1) of the formula (IV) where e is 0 and e is 1.

In the reaction with the reactive esters (2), bridges between two terminal amino radicals (formula (VI)) or between terminal and pendant amino radicals (formula (VII)) may in this case additionally be formed.

Furthermore, in addition to the aminoorganopolysiloxanes (1), it is possible to use aminoorganopolysiloxanes (1a) containing only terminal amino radicals, of the formula

(R¹O)_{f}AR_{2-f}SiO(SiR₂O)ₙSiR_{2-f}A(OR¹)_{f} (IVa)
where A, R, and R¹ are as defined above,
f is 0 or 1, and
n is an integer from 1 to 1000, preferably 50 to 1000.

Consequently, as bridges, besides the structural units of the formula (I), the precrosslinked organopolysiloxanes of the invention may contain further structural units of the formulas

SiR₂O_{1/2}—Y—SiR₂O_{1/2} (VI)

or

SiRO_{2/2}—Y—SiR₂O_{1/2} (VII)
or mixtures of (VI) and (VII), where
R and Y are as defined above.

The precrosslinked organopolysiloxanes of the invention preferably contain siloxane units of the formula (III), where d is 0 or 1, preferably 1.

The pendant and optionally terminal amino radicals A in the aminoorganopolysiloxanes (1) used in the invention or aminoorganopolysiloxanes (1a) optionally also used may possibly also react with the reactive esters (2) without forming bridges, and so the precrosslinked organopolysiloxanes may additionally contain structural units of the formulas

SiR₂O_{2/2}—R²—[NR³—R⁴]ₓ—NR³—OC—[C(Z¹)(H)]ₖ₁—[C(Z²)(H)]ₖ₂—CO—NR³—[R⁴—NR³]ₐ—R²—R₂SiO_{1/2}—[R₂SiO_{2/2}]_{b}—R₂SiO_{1/2}—R²—[NR³—R⁴]ₐ—NR³—OC—[C(Z²)(H)]ₖ₂—[C(Z¹)(H)]ₖ₁—CO₂—R⁵ (VIIIa)

and/or

SiR₂O_{1/2}—R²—[NR³—R⁴]ₓ—NR³—OC—[C(Z¹)(H)]ₖ₁—[C(Z²)(H)]ₖ₂—CO—NR³—[R⁴—NR³]ₐ—R²—R₂SiO_{1/2}—[R₂SiO_{2/2}]_{b}—R₂SiO_{1/2}—R²—[NR³—R⁴]ₐ—NR³—OC—[C(Z²)(H)]ₖ₂—[C(Z¹)(H)]ₖ₁—CO₂—R⁵ (VIIIb)
where
R, R², R³, R⁴, R⁵, k1, k2, x, a, b, Z¹, and Z² are as defined above.

In the context of this invention, formula (IV) is to be understood as meaning that p units -(SiARO)- and q units —(SiR₂O)— may be distributed in any manner, for example as a block or statistically, in the aminoorganopolysiloxane molecule.

Although not expressed respectively by the formulas (IV) and (IVa), the aminoorganopolysiloxanes (1) used in the invention and aminoorganopolysiloxanes (1a) optionally also used may also contain siloxane units selected from the group of the formulas

RSiO_{3/2}(OR¹)SiO_{3/2} and SiO_{4/2} (IXa-c)
where
R and R¹ are as defined above,
and so the precrosslinked organopolysiloxanes of the invention may also contain siloxane units of the formulas (IXa-c).

Preferably R is a monovalent, saturated or unsaturated hydrocarbon radical having 1 to 18 carbon atoms.

Examples of hydrocarbon radicals R are alkyl radicals, such as the methyl, ethyl, n-propyl, isopropyl, 1-n-butyl, 2-n-butyl, isobutyl, tert.-butyl, n-pentyl, isopentyl, neopentyl, tert.-pentyl radical; hexyl radicals, such as the n-hexyl radical; heptyl radicals, such as the n-heptyl radical; octyl radicals such as the n-octyl radical and isooctyl radicals, such as the 2,2,4-trimethylpentyl radical; nonyl radicals, such as the n-nonyl radical; decyl radicals, such as the n-decyl radical; dodecyl radicals, such as the n-dodecyl radical; octadecyl radicals, such as the n-octadecyl radical; cycloalkyl radicals, such as the cyclopentyl, cyclohexyl, cycloheptyl radical and methylcyclohexyl radicals; alkenyl radicals, such as the vinyl, 5-hexenyl, cyclohexenyl, 1-propenyl, allyl, 3-butenyl and 4-pentenyl radical; aryl radicals, such as the phenyl, naphthyl, anthryl, and phenanthryl radical; alkaryl radicals, such as o-, m-, and p-tolyl radicals; xylyl radicals and ethylphenyl radicals; and aralkyl radicals, such as the benzyl radical and the α- and ρ-phenylethyl radical.

Preferred as radical R are the methyl, ethyl, octyl, and phenyl radical, more preferably the methyl and ethyl radical.

Examples of substituted radicals R are haloalkyl radicals, such as the 3,3,3-trifluoro-n-propyl radicals, the 2,2,2,2′,2′,2′-hexafluoroisopropyl radical, the heptafluoroisopropyl radical, and haloaryl radicals, such as the o-, m-, and p-chlorophenyl radical. Further examples of substituted radicals R are polyalkyleneoxy groups, such as polyethyleneoxy, polypropyleneoxy or polyethyleneoxy/polypropyleneoxy groups.

Examples of radical R¹ are the alkyl radicals recited above for R and also the methoxyethyl, the ethoxyethyl, and the hexoxyethyl radical; preferably the radical R¹ is hydrogen, the methyl radical or the ethyl radical.

Preferably R⁵ is a C₁₋₂₀ hydrocarbon radical, which may be interrupted by one or more oxygen atoms.

Examples of radicals R apply to radicals R⁵ too.

Preferably R⁵ is a C₁₋₄ alkyl radical, such as methyl or ethyl radical.

Examples of radicals A are
—(CH₂)₃NH₂
—(CH₂)₃—NH—(CH₂)₂—NH₂
—CH₂CH(CH₃)CH₂—NH—(CH₂)₂—NH₂
—(CH₂)₃—NH(cyclohexyl)
—(CH₂)₃—NHCH₃
—(CH₂)₃—N(CH₃)₂
—(CH₂)₃—NHCH₂CH₃
—(CH₂)₃—N(CH₂CH₃)₂
—(CH₂)₄—NH₂
—CH₂CH(CH₃)CH₂—NH₂
—(CH₂)₃—NH—(CH₂)₂—NHCH₃
—(CH₂)₃—NH—(CH₂)₂—N(CH₃)₂
—(CH₂)₃—NH—(CH₂)₂—NHCH₂CH₃
—(CH₂)₃—NH—(CH₂)₂—N(CH₂CH₃)₂
—(CH₂)₃[—NH—CH₂CH_{2]2}—NH₂
and the partially or fully acetylated forms thereof, such as
—(CH₂)₃—NH(acetyl)
—(CH₂)₃—NH—(CH₂)₂—NH(acetyl), and
—(CH₂)₃—N(acetyl)-(CH₂)₂—NH(acetyl).
   Preferred examples of radicals A are:
—(CH₂)₃NH₂
—(CH₂)₃—NH—(CH₂)₂—NH₂
—CH₂CH(CH₃)CH₂—NH—(CH₂)₂—NH₂
—(CH₂)₃—NHCH₃

Preferably A is an amino radical of the formula

—R²—[NH—CH₂CH₂—]ₓNH₂
where
x is 0 or 1 and
R² is a radical of the formula —(CH₂)₃— or —CH₂—CH(CH₃)—CH₂—.

Particularly preferred examples of radical A are
—(CH₂)₃NH₂
—(CH₂)₃—NH—(CH₂)₂—NH₂ and
—CH₂CH(CH₃)CH₂—NH—(CH₂)₂—NH₂.

Further examples of a aminoorganopolysiloxanes (1) are commercially available functionalized siloxanes, such as amine oils, for example amine oils having 3-(2-aminoethyl)aminopropyl functions, and also glycol oils, phenyl oils or phenylmethyl oils that contain amino groups.

In the production of the dispersions of the invention it is possible to use one kind of aminoorganopolysiloxane (1) or different kinds of aminoorganopolysiloxane (1).

The aminoorganopolysiloxanes (1) used in producing the dispersions of the invention preferably have viscosities of 1 mPa·s to 50 000 000 mPa·s at 25° C., more preferably 50 mPa·s to 10 000 000 mPa·s at 25° C., and particularly preferably 100 mPa·s to 500 000 mPa·s at 25° C.

The aminoorganopolysiloxanes (1) used in producing the dispersions of the invention may be produced for example as described in U.S. Pat. No. 7,129,369 B2.

The dispersions of the invention preferably comprise precrosslinked
organopolysiloxanes of the invention,
emulsifiers (3), and
water (4).

The dispersions of precrosslinked organopolysiloxanes of the invention may optionally be produced using further substances that do not participate directly in the reaction.

On being dried —without addition of catalyst or change in pH—the dispersions of the invention form a silicone network, preferably an elastic silicone network. The dispersions of the invention preferably form elastomeric films following removal of the water.

In the process of the invention—the reaction of aminoorganopolysiloxanes (1) and optionally (1a) with reactive esters (2) in accordance with the invention—preferably no metal-containing catalysts are used.

The dispersions of the invention therefore preferably contain no catalysts.

In the production of the precrosslinked organopolysiloxanes of the invention, aminoorganopolysiloxanes (1) and reactive esters (2) are used, and these components react with one another preferably at room temperature. No metal-containing, additional catalysts are needed to assist this reaction, and therefore preferably no transition metals from subgroup VIII of the periodic table, and compounds thereof, and no metals from main groups Ill, IV, and V of the periodic table, and compounds thereof, are used; in this definition, the elements C, Si, N, and P are not deemed to be metals.

Furthermore, the reaction proceeds preferably in the neutral range, i.e. in the pH range from approx. 4 to 8, which is obtained by the components themselves. The high reactivity means there is also no need for a specifically controlled chemical reaction, and preferably not for any heating either.

In the production of the dispersions of the invention it is possible to use one kind of aminoorganopolysiloxane (2) as crosslinker or different kinds of aminoorganopolysiloxanes (2) as crosslinkers.

Preferred esters (2) used are oxalamidoester-terminated organopolysiloxanes of the formula

R⁵—O₂C—CO—NH—R²—R₂SiO_{1/2}—[R₂SiO_{2/2}]_{b}—R₂SiO_{1/2}—R²—NH—OC—CO₂—R⁵
where
R is as defined above,
R² is a radical of the formula —(CH₂)₃— or —CH₂—CH(CH₃)—CH₂—,
R⁵ is as defined above, preferably a C₁₋₄ alkyl radical, especially a methyl or ethyl radical, and
b is 0 or an integer from 1 to 500, preferably 20 to 350.

Y is therefore preferably a radical of the formula

—R²[NH—CH₂CH₂]ₓ—NH—OC—CO—NH—R²—R₂SiO_{1/2}—[R₂SiO_{2/2}]_{b}—R₂SiO_{1/2}—R²—NH—OC—CO—NH—[CH₂CH₂—NH]ₓ—R²—
where x is 0 or 1,
b and R are as defined above, and
R² is a radical of the formula —(CH₂)₃— or —CH₂—CH(CH₃)—CH₂—.

Depending on the use of crosslinker (2) or of linear, branched or resin-like aminoorganopolysiloxanes (1), the precrosslinked organopolysiloxanes may have branched or even highly branched or highly crosslinked structures with linear fractions.

In the process of the invention, the nature and amount of aminoorganopolysiloxanes and esters are selected such that the organopolysiloxanes in the resulting dispersions are precrosslinked.

Preferred aminoorganopolysiloxanes (1) used are those that contain on average at least one primary amino function in the pendant amino radicals A, thus affording precrosslinked organopolysiloxanes of the invention. In addition it is also possible to use aminoorganopolysiloxanes (1a) that contain only terminal amino radicals A with primary amino functions.

When aminoorganopolysiloxanes (1a) are used, they are used preferably in amounts of 10 to 300 parts by weight per 100 parts by weight of aminoorganopolysiloxanes (1).

Furthermore, the use of mixtures of aminoorganopolysiloxanes (1) and unfunctional organopolysiloxanes (1 b) also affords precrosslinked organopolysiloxanes of the invention.

Unfunctional organopolysiloxanes (1 b) here are organopolysiloxanes without amino radicals A, preferably linear organopolysiloxanes of the general formula

R⁷ᵤR⁶₃₋ᵤSiO[R⁶₂SiO]ᵥSiR⁶₃₋ᵤR⁷ᵤ (X)
where R⁶ is as defined for R and
R⁷ is as defined for R⁶ or is a HO— group,
u is 0 or 1, and
v is 0 or an integer from 1 to 2000.
Preferably, R⁶ is a C₁₋₁₈ hydrocarbon radical, preferably a C₁₋₁₈ alkyl radical.

Examples of unfunctional organopolysiloxanes (1 b) are dialkylpolysiloxanes, preferably dimethylpolysiloxanes.

When unfunctional organopolysiloxanes (1 b) are used, they are used preferably in amounts of 100 to 800 parts by weight per 100 parts by weight of aminoorganopolysiloxanes (1).

The degree of crosslinking here is dependent on the employed ratio of equivalents of —OR⁵ in the reactive esters (2) to the amino radical A in the aminoorganopolysiloxane (1).

For producing the dispersions of the invention composed of aminoorganopolysiloxane (1) and reactive ester (2), the ester (2) is used preferably in amounts of 0.1 to 10 equivalents of —OR⁵, more preferably 0.2 to 5 equivalents of —OR⁵, particularly preferably 0.3 to 3 equivalents of —OR⁵, per equivalent of amino radical A in the aminoorganopolysiloxane (1).

The dispersions of precrosslinked organopolysiloxanes of the invention are produced by intensive mixing of aminoorganopolysiloxanes (1) with esters (2), emulsifiers (3), and water (4) with one another.

The production may take place batchwise or continuously.

The way in which the components used for producing the dispersions of the invention are mixed is not especially critical, and may be executed in different order.

For example, components (1) and (2) may be premixed with one another, then the emulsifier(s)added, followed by the incorporation of water (4). Another possibility is to meter components (1) to (4) sequentially into the emulsifying apparatus. In special cases it may be advantageous—for example because of the viscosity or reactivity of the employed aminoorganopolysiloxanes (1) and optionally (1a) and optionally unfunctional organopolysiloxanes (1b)—to mix ester (2) with an aminoorganopolysiloxane and thereafter to incorporate a different aminoorganopolysiloxane or unfunctional organopolysiloxane, or vice versa, depending on which achieves the more favorable rheological properties for the processing of the components.

Furthermore, it is also possible to introduce the ester (2) as crosslinker into the finished emulsion of aminoorganopolysiloxanes (1), in order thereby to achieve the desired reaction and crosslinking of the aminoorganopolysiloxane in the emulsion. To obtain VOC-free products, these being products without volatile organic compounds, the byproduct alcohol R⁵OH (where R⁵ is as defined above) can be completely or partially removed by suitable known measures such as distillation, membrane methods, or other separation methods.

In the production of the dispersions of the invention, water (4) is used in amounts of preferably 1% to 99% by weight, more preferably 25% to 95% by weight, in each case based on the total weight of all the ingredients of the dispersion.

The aqueous dispersion of precrosslinked organopolysiloxanes of the invention is used as an oil-in-water system.

As emulsifiers (3) for producing the aqueous dispersions of precrosslinked organopolysiloxanes it is possible to use all anionic, nonionic, cationic or amphoteric emulsifiers known to date, both individually and as mixtures of different emulsifiers, with which it has also previously been possible to produce aqueous dispersions, more particularly aqueous emulsions of organopolysiloxanes.

### Examples of Anionic Emulsifiers are as Follows:

1. Alkyl sulfates, particularly those having a chain length of 8 to 18 carbon atoms, alkyl and alkaryl ether sulfates having 8 to 18 carbon atoms in the hydrophobic radical, and 1 to 40 ethylene oxide (EO) and/or propylene oxide (PO) units.
2. Sulfonates, particularly alkyl sulfonates having 8 to 18 carbon atoms, alkylaryl sulfonates having 8 to 18 carbon atoms, taurides, esters and monoesters of sulfosuccinic acid with monohydric alcohols or alkylphenols having 4 to 15 carbon atoms; these alcohols or alkylphenols may optionally also be ethoxylated with 1 to 40 EO units.
3. Alkali metal and ammonium salts of carboxylic acids having 8 to 20 carbon atoms in the alkyl, aryl, alkaryl or aralkyl radical.
4. Partial esters of phosphoric acid and the alkali metal salts and ammonium salts of thereof, particularly alkyl and alkaryl phosphates having 8 to 20 carbon atoms in the organic radical and alkyl ether phosphates and alkaryl ether phosphates having 8 to 20 carbon atoms in the alkyl or alkaryl radical and 1 to 40 EO units.

### Examples of Nonionic Emulsifiers are as Follows:

5. Polyvinyl alcohol still having 5% to 50%, preferably 8% to 20%, of vinyl acetate units and having a degree of polymerization of 500 to 3000.
6. Alkyl polyglycol ethers, preferably those having 3 to 40 EO units and alkyl radicals of 8 to 20 carbon atoms.
7. Alkylaryl polyglycol ethers, preferably those having 5 to 40 EO units and 8 to 20 carbon atoms in the alkyl and aryl radicals.
8. Ethylene oxide/propylene oxide (EO/PO) block copolymers, preferably those having 8 to 40 EO or PO units.
9. Addition products of alkylamines having alkyl radicals of 8 to 22 carbon atoms with ethylene oxide or propylene oxide.
10. Fatty acids having 6 to 24 carbon atoms.
11. Alkyl polyglycosides of the general formula R″—O—Zₒ, where R″ denotes a linear or branched, saturated or unsaturated alkyl radical having on average 8-24 carbon atoms and Zₒ an oligoglycoside radical having on average o=1-10 hexose or pentose units or mixtures thereof.
12. Natural substances and derivatives thereof, such as lecithin, lanolin, saponins, cellulose; cellulose alkyl ethers and carboxyalkyl celluloses, the alkyl groups of which have in each case up to 4 carbon atoms.
13. Linear organo(poly)siloxanes containing polar groups containing in particular the elements O, N, C, S, P, Si, especially those having alkoxy groups with up to 24 carbon atoms and/or up to 40 EO and/or PO groups.

### Examples of Cationic Emulsifiers are as Follows:

14. Salts of primary, secondary, and tertiary fatty amines having 8 to 24 carbon atoms with acetic acid, sulfuric acid, hydrochloric acid, and phosphoric acids.
15. Quaternary alkylammonium and alkylbenzeneammonium salts, especially those in which the alkyl groups have 6 to 24 carbon atoms, especially the halides, sulfates, phosphates, and acetates.
16. Alkylpyridinium, alkylimidazolinium, and alkyloxazolinium salts, especially those in which the alkyl chain has up to 18 carbon atoms, specifically the halides, sulfates, phosphates, and acetates.

Particularly suitable amphoteric emulsifiers are as follows:
17. Amino acids having long-chain substituents, such as N-alkyldi(aminoethyl)glycine or salts of N-alkyl-2-aminopropionic acid.
18. Betaines, such as N-(3-acylamidopropyl)-N,N-dimethylammonium salts with a C₈-C₁₈ acyl radical, and alkylimidazolinium betaines, or quaternized alkyl or substituted alkyl derivatives of N,N-dimethylglycine.

Preferred emulsifiers for producing organopolysiloxanes precrosslinked for aqueous dispersions are nonionic emulsifiers, especially the alkyl polyglycol ethers listed under 6. above.

Constituent (3) may consist of one of the abovementioned emulsifiers or a mixture of two or more abovementioned emulsifiers; it may be used in pure form or in the form of solutions of one or more emulsifiers in water or organic solvents.

In the production of the dispersions of the invention, the emulsifiers (3) are used in amounts of preferably 0.1% to 60% by weight, more preferably 0.5% to 30% by weight, in each case based on the total weight of aminoorganopolysiloxanes (1) and ester (2).

If the aminoorganopolysiloxane (1) or the ester (2), or the precrosslinked organopolysiloxane obtained is itself acting as an emulsifier, there is no need to add separate emulsifier (3).

The emulsifying process for producing the dispersion is carried out preferably at temperatures below 120° C., more preferably at 5° C. to 100° C., particularly preferably at 10° C. to 80° C. The increase in temperature comes about preferably through the introduction of mechanical shear energy, which is needed for the emulsifying process. The increase in temperature is not needed in order to accelerate a chemical process. In addition, the dispersions are produced preferably at the pressure of the ambient atmosphere, but may also be produced at higher or lower pressures.

The reaction of aminoorganopolysiloxanes (1) with esters (2) in the production of the dispersions preferably proceeds in a few minutes to several days.

The alcohols obtained as condensation byproducts in the production of the dispersions may remain in the product or else be removed, for example by vacuum distillation, membrane methods, or by extraction.

The average particle size measured by laser diffraction (using the Mastersizer 3000 particle size analyzer from Malvern Panalytical) in the dispersions of the invention is preferably in the range from 0.001 to 50 μm, preferably 0.005 to 20 μm, particularly preferably in the range from 0.01 to 10 μm.

The pH may vary from 1 to 14, preferably 3 to 9, more preferably 4 to 8.

The invention provides cosmetic compositions comprising aqueous dispersions, preferably aqueous emulsions, of precrosslinked organopolysiloxanes.

The cosmetic composition of the invention comprises aqueous dispersions of precrosslinked organopolysiloxanes preferably in amounts of from 0.2% to 65% by weight, more preferably from 0.5% to 50% by weight, in each case based on the total weight of the cosmetic composition.

The cosmetic compositions of the invention preferably comprise water as a cosmetically acceptable medium.

The cosmetic composition of the invention preferably comprises a conditioning agent. The term conditioning agents is used, in accordance with K. Krummel, Stephane Chiron, J. Jachowicz, Chapter 14, in: “The Chemistry and Manufacture of Cosmetics”, volume II, Formulating, third edition by Mitchell L. Schlossmann, 2000, pp. 359-396, to refer to cosmetic ingredients that modify the hair surface and influence the condition of the hair. Cosmetic compositions comprising conditioning agents are employed in order to modify or improve the softness of the hair, greater ease of disentangling, reduction in wet and dry combing force, care of the hair, prevention of electrostatic charging, easier sliding effect through the hair and along the hair surface, improvement in hair shine, retention of color fastness of hair, reduction in hair breakage, retention of hair shape, and further cosmetic properties associated with natural and healthy hair.

The cosmetic composition of the invention improves one or more of the effects stated above, especially the combability and the deposition of the silicone on the hair.

Examples of conditioning agents and their INCI names are described in the “International Cosmetic Ingredient Dictionary & Handbook” of the Personal Care Product Council (ed.).

For reference it is possible to use the worldwide web-based “wINCI Web Based International Cosmetic Ingredient Dictionary & Handbook (http://online.personalcarecouncil.org/jsp/Home.jsp) or the International Cosmetic Ingredient Dictionary & Handbook, 13th Edition, The Personal Care Products Council (formerly: The Cosmetic, Toiletry, and Fragrance Association (CTFA)), 2010.

Conditioning agents used are preferably those selected from the group of cationic polymers,
cationic surfactants,
nonpolymeric quaternary ammonium compounds,
organopolysiloxanes and organopolysiloxane copolymers different from the precrosslinked organopolysiloxanes containing structural units of the formula (I),
fatty acid esters and fatty acid alcohols,
natural or synthetic oils and waxes, and
panthenol, lipids, proteins, and hydrolyzed proteins,
and mixtures thereof.

Preferred examples of conditioning agents are cationic polymers. These are understood as meaning polymers bearing pendant or terminal cationic groups or bearing pendant or terminal groups that can be converted into a cationic group by ionization.

Preference is given to using cationic polymers that have a quaternary ammonium group.

Examples of cationic polymers preferably employed are published in the International Cosmetic Ingredient Dictionary & Handbook under the designation Polyquaternium, with each polymer being identified by an individual numerical abbreviation, for example Polyquaternium-1.

Further examples of cationic polymers are quaternary ammonium group-containing derivatives of modified polysaccharides, for example polymers having the INCI name Cassia Hydroxypropyltrimonium Chloride, derivatives of modified cellulose and/or starch, for example a quaternary ammonium derivative of a propylene glycol ether-modified Cyamopsis Tetragonoloba (guar) gums having the INCI name Guar Hydroxypropyltrimonium Chloride, or polymeric quaternary ammonium salts of the reaction product of hydroxyethylcellulose with a trimethylammonium-substituted epoxide, such as cellulose 2-hydroxyethyl 2-(2-hydroxy-3-(trimethylammonium)propoxy)ethyl 2-hydroxy-3-(trimethylammonio)propyl ether chloride, such as cellulose 2-hydroxyethyl 2-hydroxy-3-(trimethylammonium)propyl ether chloride, such as cellulose 2-hydroxyethyl 2-hydroxy-3-(trimethylammonium)propyl ether chloride, such as cellulose 2-[2-hydroxy-3-(trimethylammonium)propoxy]ethyl ether chloride, such as cellulose 2-[2-hydroxy-3-trimethylammonium)propoxy]ethyl ether chloride having the INCI name Polyquaternium-10.

Further examples of cationic polymers are quaternary ammonium group-containing acrylic acid polymer derivatives, acrylic acid copolymer derivatives, methacrylic acid derivatives, and methacrylic acid copolymer derivatives, for example polymers having the INCI name Polyquaternium-37.

Further examples of cationic polymers are quaternary ammonium group-containing copolymers of dimethyldiallylammonium chloride and acrylic acid, for example polymers having the INCI name Polyquaternium-22.

Further examples of cationic polymers are quaternary ammonium group-containing copolymers of derivatives of vinylpyrrolidone, vinylimidazole, and vinylimidazoline and methacrylic acid, for example polymers having the INCI name Polyquaternium-86.

Further examples of cationic polymers are quaternary ammonium group-containing copolymers of acrylamide and dimethyldiallylammonium chloride, for example polymers having the INCI name Polyquaternium-7.

Further examples of cationic polymers are quaternary ammonium group-containing copolymers of the reaction product of diethyl sulfate with vinylpyrrolidone and dimethylaminoethyl methacrylate, for example polymers having the INCI name Polyquaternium-11.

When cationic polymers are used, the cosmetic composition of the invention contains cationic polymers preferably in amounts of from 0.01% to 5% by weight, more preferably from 0.05% to 4% by weight, particularly preferably 0.10% to 3% by weight, in each case based on the total weight of the cosmetic composition.

Further preferred examples of conditioning agents are cationic surfactants. Examples of cationic surfactants used with preference correspond to the materials listed in points 14. to 16. under examples of cationic emulsifiers. Examples are cetyltrimethylammonium salts or behenyltrimethylammonium salts. The anionic counterion present may for example be chloride, bromide, methosulfate.

INCI names of cationic surfactants used with preference are for example Cetrimonium Chloride, Cetrimonium Methosulfate, Behentrimonium Chloride, Behentrimonium Methosulfate, Steartrimonium Bromide.

When cationic surfactants are used, the cosmetic composition of the invention contains cationic surfactants preferably in amounts of from 0.1% to 7% by weight, more preferably from 0.15% to 6% by weight, particularly preferably 0.2% to 5% by weight, in each case based on the total weight of the cosmetic composition.

Further examples of conditioning agents are nonpolymeric quaternary ammonium compounds. This term refers to nonpolymeric ammonium compounds that are present in cationic form or can be converted into a cationic group by ionization.

Examples of nonpolymeric quaternary ammonium compounds used with preference are dimethyldioctadecylammonium chloride having the INCI name Distearyldimonium Chloride, N-[3-(dimethylamino)propyl]octadecaneamide having the INCI name Stearamidopropyl Dimethylamine, or compounds having the INCI name Dicocoylethyl Hydroxyethylmonium Methosulfate or Quaternium-87.

Further preferred examples of conditioning agents are organopolysiloxanes and organopolysiloxane copolymers that are different from the precrosslinked organopolysiloxanes having structural units of the formula (I) that are present in the aqueous dispersions. The organopolysiloxanes may be present in the form of an oil, wax, gum or resin or in the form of an emulsion.

Examples of such organopolysiloxanes different from the precrosslinked organopolysiloxanes having structural units of the formula (I) are as follows:

Cyclic organopolysiloxanes of the formula

[R*₂SiO]_{x′},
where x′ is an integer from 4 to 8,
linear organopolysiloxanes of the general formula

R*₃SiO[R*₂SiO]_{y}SiR*₃ or

HOSiR*₂O[R*₂SiO]_{y}SiR*₂OH,
where y is 0 or an integer from 1 to 2000,
and resin-like organopolysiloxanes of the general formula

R*ₜSiO_{(4-t)/2}
where R* is in each case as defined above for R, R¹ or A, preferably for R and R¹, and
t is 0, 1, 2 or 3,
and so the organopolysiloxane resin is constructed of M, D, T and/or Q units, with preference being given both to a combination primarily or exclusively of D and T units and to a combination primarily or exclusively of M and Q units; in the case of the resins constructed primarily or exclusively of D and T units, T units are present preferably in a molar ratio T/[M+D+T+Q] of 0.45 to 1, more preferably of 0.55 to 1.0, and the number of the M and Q units in both cases is preferably zero, and, in the case of the organopolysiloxane resins constructed primarily or exclusively of M and Q units, Q units are present preferably in a molar ratio Q/[M+D+T+Q] of 0.25 to 0.9, more preferably of 0.35 to 0.7, and the number of the D and T units in both cases is preferably zero.

Examples of organopolysiloxanes present in the form of an oil are polydimethylsiloxanes having a viscosity of 0.65 to 2 000 000 mPas (25° C.) and the INCI designations Disiloxane and Dimethicone.

Further examples of organopolysiloxanes present in the form of an oil or wax are functionalized organopolysiloxanes, for example polyalkysiloxanes where at least one alkyl radical is different from methyl, for example organopolysiloxanes having the INCI name Stearyl Dimethicone, Cetyl Dimethicone or C₂₆₋₂₈ Alkyl Dimethicone, or, for example, polyarylsiloxanes and polyarylalkylsiloxanes, for example organopolysiloxanes having the INCI name Phenyl Trimethicone, Trimethylsiloxyphenyl Dimethicone or Dimethylphenyl Dimethicone, or, for example, organopolysiloxanes having an organofunctional radical such as an aminopropyl, aminopropylaminoethyl, aminopropylaminoisobutyl radical, for example organopolysiloxanes having the INCI name Amodimethicone, or, for example, organopolysiloxanes having a polyethylene glycol or polyalkylene glycol radical, for example organopolysiloxanes having the INCI name PEG 12 Dimethicone, PEG/PPG-25,25-Dimethicone or Cetyl PEG/PPG-15/15 Butyl Ether Dimethicone.

Further examples of organopolysiloxanes are silicone resins having the INCI names Trimethylsiloxysilicate or Polymethylsilsesquioxane.

When such organopolysiloxanes or organopolysiloxane copolymers are used, the cosmetic composition of the invention comprises organopolysiloxanes and organopolysiloxane copolymers that are different from the precrosslinked organopolysiloxanes having structural units of the formula (I) present in the aqueous dispersions, in amounts preferably of from 0.1% to 40% by weight, more preferably from 0.2% to 30% by weight, particularly preferably from 0.3% to 20% by weight, in each case based on the total weight of the cosmetic composition.

Further preferred examples of conditioning agents are fatty acid esters and fatty acid alcohols.

Examples of fatty acid alcohols are alcohols having C8-C28 carbon chains, such as the fatty alcohols 1-octadecanol having the INCI name Stearyl Alcohol, 1-hexadecanol having the INCI name Cetyl Alcohol, or fatty alcohols having the INCI names Cetearyl Alcohol, Myristyl Alcohol, Caprylic Alcohol, Lauryl Alcohol, Decyl Alcohol, and Oleyl Alcohol.

In addition to conditioning properties, fatty acid alcohols also fulfill a structuring, thickening effect in cosmetic compositions.

Further examples of fatty acid esters are esters of the fatty acids having the INCI name Palmitic Acid, Oleic Acid, Linolic Acid, Linoleic Acid, Caprylic Acid, Myristic Acid, Stearic Acid, for example fatty acid esters having the INCI name Isopropyl Palmitate, Ethylhexyl Palmitate, Isopropyl Myristate, Isopropyl Stearate.

When fatty acid esters and fatty acid alcohols are used, the cosmetic composition of the invention contains fatty acid esters and fatty acid alcohols preferably in amounts of from 0.1% to 15% by weight, more preferably from 0.3% to 12% by weight, particularly preferably from 0.5% to 10% by weight, in each case based on the total weight of the cosmetic composition.

Further preferred examples of conditioning agents are natural or synthetic oils and waxes.

Examples of preferred oils and waxes are hydrocarbons having linear or branched, saturated or unsaturated C4-C60 carbon chains, such as oils and waxes having the INCI names Isododecane, hydrated Polyisobutylene, hydrated Polydecene, Paraffin and Isoparaffin.

Further examples of preferred oils and waxes are carnauba wax, beeswax, wool wax, microcrystalline wax, jojoba oil, rice oil, calendula oil, sunflower oil, soybean oil, coconut oil, olive oil, and almond oil.

When natural or synthetic oils and waxes are used, the cosmetic composition of the invention contains oils and waxes preferably in amounts of from 0.1% to 10% by weight, more preferably from 0.2% to 7% by weight, particularly preferably from 0.3% to 5% by weight, in each case based on the total weight of the cosmetic composition.

Further preferred examples of conditioning agents are panthenol, lipids, such as ceramides, proteins, and hydrolyzed proteins, such as hydrolyzed collagen, hydrolyzed wheat proteins, and hydrolyzed silk.

The cosmetic composition optionally comprises further cosmetically customary additives such as, for example, surfactants, thickeners, gelling agents, film formers, moisturizing agents, UV filters, pearlescent pigments, vitamins, antioxidants, caffeine, anti-dandruff active ingredients or preservatives.

Examples of further additives customary in cosmetology and their INCI names are described in the “International Cosmetic Ingredient Dictionary & Handbook” of the Personal Care Product Council.

The cosmetic composition optionally comprises further cosmetically customary additives such as surfactants.

Examples of surfactants customary in cosmetology are also described in K. Schrader, A. Domsch, Cosmetology-Theory and Practice, volume II, pages II-8 to II-22, Verlag für chemische Industrie, 2005, and also in points 1. to 18. under examples of emulsifiers.

Examples of anionic surfactants used with preference correspond to the materials listed in points 1. to 3. under examples of anionic emulsifiers.

INCI names of anionic surfactants used with preference are for example Sodium Lauryl Sulfate, Ammonium Laureth Sulfate, Sodium Laureth Sulfate, Disodium 2-Sulfolaurate, Disodium Lauryl Sulfosuccinate or Disodium Laureth-Sulfosuccinate.

When anionic surfactants are used, the cosmetic composition of the invention contains anionic surfactants preferably in amounts of from 1% to 30% by weight, more preferably from 5% to 25% by weight, particularly preferably 7% to 20% by weight, in each case based on the total weight of the cosmetic composition.

Examples of nonionic surfactants used with preference correspond to the materials listed in points 5. to 13. under examples of nonionic emulsifiers.

INCI names of nonionic surfactants used with preference are for example Coco Glucoside, Lauryl Glucoside, Decyl Glucoside, PEG-40 Hydrogenated Castor Oil, Polysorbate 80 or PEG-7 Glyceryl Cocoate.

When nonionic surfactants are used, the cosmetic composition of the invention contains nonionic surfactants preferably in amounts of from 1% to 15% by weight, more preferably from 2% to 12% by weight, particularly preferably 3% to 10% by weight, in each case based on the total weight of the cosmetic composition.

Examples of amphoteric surfactants used with preference correspond to the materials listed in points 17. and 18. under examples of nonionic emulsifiers. Further preferred examples are compounds from the alkylamidobetaine, alykylamphoacetate, and alkylamphopropionate classes.

INCI names of nonionic surfactants used with preference are for example Cocamidopropyl Betaine, Cetyl Betaine, Cocamide MEA, Cocamide DEA, Cocamide MIPA, Sodium Cocoamphoacetate or Sodium Cocoamphopropionate.

When amphoteric surfactants are used, the cosmetic composition of the invention contains amphoteric surfactants preferably in amounts of from 1% to 15% by weight, more preferably from 2% to 12% by weight, particularly preferably 3% to 10% by weight, in each case based on the total weight of the cosmetic composition.

The cosmetic composition optionally comprises further cosmetically customary additives such as thickeners.

Examples of thickeners used with preference are modified polysaccharides such as starch, cellulose, gum arabic, and guar gums, for example polymers having the INCI name Cellulose Gum, Guar Gum, Xanthan Gum or Cassia Gum.

Further examples of thickeners are hydrophobically modified nonionic cellulose derivatives, for example the cellulose derivative having the INCI name Hydroxyethylcellulose.

Further examples of thickeners are crosslinked acrylic acid and methacrylic acid polymers and derivatives of crosslinked acrylic acid and methacrylic acid polymers, for example polymers having the INCI name Carbomer.

Further examples of thickeners are agents that achieve a thickening effect in combination with surfactants. Examples are monoglycerides of fatty acids, mono/diglycerides of ethoxylated fatty acids, and ethoxylated fatty alcohols. INCI names of thickeners used with preference that achieve a thickening effect in combination with surfactants are PEG-120 Methyl Glucose Dioleate, PEG-150 Distearate, Myristyl Glycol, PEG-200 Glyceryl Palmitate, Laureth-4 or PEG-200 Glyceryl Palmitate.

Further examples of thickeners are salts, for example salts having the INCI name Sodium Chloride.

When thickeners are used, the cosmetic composition of the invention comprises thickeners preferably in amounts of 0.1% to 10% by weight, in each case based on the total weight of the cosmetic composition.

The cosmetic composition optionally comprises further cosmetically customary additives such as film formers.

Preferred examples of film formers are polymers.

Examples of film-forming polymers used with preference are described in the “International Cosmetic Ingredient Dictionary & Handbook” of the Personal Care Product Council.

Examples of preferred film-forming polymers are acrylic acid polymer derivatives, acrylic acid copolymer derivatives, methacrylic acid derivates, and methacrylic acid copolymer derivatives.

Examples of preferred anionic polymers are copolymers of vinyl acetate and one or more acrylic acid monomers, methacrylic acid monomers or esters thereof, for example polymers having the INCI name Acrylates/VA Copolymer.

Further examples of preferred film-forming polymers are copolymers of vinylpyrrolidone and one or more acrylic acid monomers, methacrylic acid monomers or esters thereof, for example polymers having the INCI name Acrylates/VP Copolymer.

Further examples of preferred film-forming polymers are copolymers of tert-butylacrylamide and one or more acrylic acid monomers, methacrylic acid monomers or esters thereof, for example polymers having the INCI name Acrylates/t-Butylacrylamide Copolymer.

Further examples of preferred film-forming polymers are copolymers of vinyl acetate, crotonic acid, and vinyl neodecanoate monomers, for example polymers having the INCI name VA/Crotonates/Vinyl Neodecanoate Copolymer.

Further examples of preferred film-forming polymers are copolymers of vinyl acetate, crotonic acid, and vinyl neodecanoate monomers, for example polymers having the INCI name Crotonic Acid/Vinyl C8-C12 Isoalkyl Esters/VA/Bis-Vinyldimethicone Copolymer.

When film-forming polymers are used, the cosmetic composition of the invention contains film-forming polymers preferably in amounts of amounts of from 0.1% to 15% by weight, more preferably from 0.2% to 10% by weight, particularly preferably 0.3% to 7% by weight, in each case based on the total weight of the cosmetic composition.

The cosmetic composition optionally comprises further cosmetically customary additives such as moisturizing agents.

Examples of moisturizing agents used with preference are glycerol, sorbitol, xylitol, polyethylene glycol, propane-1,2-diol, propane-1,3-diol or polypropylene glycol.

When moisturizing agents are used, the cosmetic composition of the invention contains moisturizing agents preferably in amounts of amounts of from 0.1% to 10% by weight, more preferably from 0.2% to 8% by weight, particularly preferably 0.3% to 6% by weight, in each case based on the total weight of the cosmetic composition.

The cosmetic composition optionally comprises further cosmetically customary additives such as pearlizing agents.

Examples of pearlizing agents used with preference are pearlescent pigments or glycol distearate.

When agents imparting pearly luster are used, the cosmetic composition of the invention contains agents imparting pearly luster preferably in amounts of from 0.1% to 7% by weight, more preferably from 0.2% to 6% by weight, particularly preferably 0.3% to 5% by weight, in each case based on the total weight of the cosmetic composition.

The cosmetic compositions are produced preferably by mixing at least one aqueous dispersion of precrosslinked organopolysiloxanes of the invention with optionally at least one conditioning agent and optionally further cosmetically customary additives in a cosmetically acceptable medium, preferably water.

The individual ingredients may be mixed with one another in a hot/hot, hot/cold or cold/cold process.

In the production of the cosmetic composition of the invention, the dispersions of precrosslinked organopolysiloxanes of the invention are added preferably at temperatures of at most 50° C., more preferably at temperatures of at most 40° C., particularly preferably at temperatures of at most 35° C. They are added preferably at temperatures of at least 5° C., more preferably at temperatures of at least 10° C.

The cosmetic composition of the invention may be present in the form of an emulsion, a suspension, a solution, a cream, a lotion, a mousse, a stick, a soap bar, a paste, or a gel.

The cosmetic composition of the invention in the form of an emulsion may be present in the form of a W/O emulsion (water-in-oil emulsion), an O/W emulsion (oil-in-water emulsion) or as a multiple emulsion.

When the aim is to produce a cosmetic composition comprising an aqueous dispersion of precrosslinked organopolysiloxanes of the invention in the form of an emulsion having a translucent or transparent appearance, then preference is given to using aqueous dispersions of precrosslinked organopolysiloxanes of the invention having particle sizes <700 nm, more preferably having particle sizes <400 nm, especially preferably having particle sizes <300 nm.

The invention further provides for the use of the cosmetic composition of the invention for treating keratin fibers, such as hair. The cosmetic compositions are used preferably for cleansing and caring for keratin fibers, such as hair, or for shaping keratin fibers, such as hair.

Examples of products for the cleansing and care of hair are hair shampoos, hair rinses (rinse-off conditioners), hair treatments, hair masks, hair sera, hair mousses, hair styling sprays, hair creams, hair gels, hair oils, hair end fluids, and hair colorants.

The invention further provides for the use of the cosmetic composition of the invention for conditioning keratin fibers, such as hair, for the purpose in particular of making keratin fibers easier to comb.

The invention further provides a process for treating keratin fibers, preferably hair, by applying the cosmetic compositions of the invention to the keratin fibers, preferably hair, and then optionally rinsing with water.

In the examples described below all reported amounts in parts and percentages are based on weight unless otherwise stated. In addition, all viscosity data relate to a temperature of 25° C. Unless otherwise stated, the examples that follow are executed at the pressure of the ambient atmosphere, i.e. about 1020 hPa, and at room temperature, i.e. at about 20° C., or at a temperature that results when combining the reactants at room temperature without supplemental heating or cooling.

### Synthesis of oxalamido-ethyl-ester-terminated organopolysiloxanes S1-S2

### Synthesis of the oxalamido-ethyl-ester-terminated organopolysiloxane S1

An apparatus consisting of a 4-1 three-necked flask with magnetic stirrer bar, magnetic stirrer, reflux condenser, internal thermometer, and dropping funnel was inertized with nitrogen gas. The flask was then initially charged under a continuous stream of nitrogen with 2679 g (18.45 mol) of diethyl oxalate (M=146.14 g/mol; CAS No. 95-92-1; available from Sigma-Aldrich, Munich, Germany) and to this was added dropwise with continuous stirring 600 g (4.07 mol) of (3-aminopropyl)dimethylmethoxysilane (M=147.29 g/mol; CAS No. 31024-26-7; available from Gelest, Morrisville, USA) at a rate such that the temperature of the reaction mixture remained below 50° C. throughout. This afforded a slightly yellowish clear liquid, which was stirred at room temperature for a further hour after the end of the addition. The excess diethyl oxalate was then distilled off under reduced pressure (2 mbar). 1995 g of diethyl oxalate (13.65 mol) was obtained as a colorless, clear liquid. The liquid distillation residue was fractionally distilled at an internal temperature of 160° C. In the boiling range of 136 to 141° C./2 mbar vacuum, 949 g (3.84 mol) of the desired main product ethyl 2-((3-(methoxydimethylsilyl)propyl)amino)-2-oxoacetate (EtO—CO—CO—HN—CH₂CH₂CH₂—Si—Me₂(OMe)) P1 (M=247.37 g/mol) distilled as a colorless, clear liquid. The yield was 94% based on the (3-aminopropyl)dimethylmethoxysilane used.

A 2-1 round-bottomed flask was initially charged with 164.95 g (0.66 mol) of P1 and 740 g (10.00 mol Si) of α,ω-OH-terminated linear dimethylsiloxane (CAS No. 70131-67-8) having a viscosity of 50 mPas. The round-bottomed flask containing the reaction mixture was heated on a rotary evaporator in a 130° C. oil heating bath at a vacuum of 6 mbar for 30 minutes under rotation. The vacuum was then released, 0.02 g of a 40% solution of PNCl₂ in ethyl acetate was added, and the mixture was heated under reduced pressure for one hour on the rotary evaporator (130° C. oil bath, 6 mbar vacuum). The vacuum was then released again, 0.02 g of a 40% solution of PNCl₂ in ethyl acetate (10 ppm PNCl₂) was added, and the mixture was heated under reduced pressure for a further hour on the rotary evaporator (130° C. oil bath, 6 mbar vacuum). The vacuum was then released once again, 0.02 g of a 40% solution of PNCl₂ in ethyl acetate (10 ppm PNCl₂) was added, and the mixture was heated under reduced pressure for 4 hours on the rotary evaporator (130° C. oil bath, 6 mbar vacuum). After cooling to room temperature, 8 g of magnesium oxide (heavy MgO; CAS No. 1309-48-4; available from Sigma-Aldrich, Munich, Germany) was added to the flask contents and the mixture was stirred at room temperature for 1 h. The suspension was then filtered under 6 mbar pressure through a Seitz© T-120 depth filter (available from Pall Corporation, New York, USA). This afforded 793 g of a colorless, clear oil having a viscosity of ˜95 mPas.

### Synthesis of the oxalamido-ethyl-ester-terminated organopolysiloxane S2

A stirred-flask apparatus consisting of 1-1 three-necked flask with heating mantle, mechanical stirrer, internal thermometer, and water separator with reflux condenser was initially charged with 400 g (5.41 mol Si) of α,ω-OH-terminated linear dimethylsiloxane (CAS No. 70131-67-8) having a viscosity of 50 mPas and 19.8 g (0.08 mol) of P1 and the mixture was heated to 80° C. while stirring. After adding 50 mg of PNCl₂ (100%), the pressure was reduced (10 mbar) and the flask contents were heated to 100° C. The water that formed was removed via the water separator. After 5 minutes, the vacuum was adjusted to 40 mbar and the mixture stirred for 30 minutes under these conditions. The vacuum was then released, another 25 mg of PNCl₂ (100%) added, and the mixture heated at 100° C. and 40 mbar while stirring. The vacuum was then released again, 25 mg of PNCl₂ (100%) added, and the mixture heated further at 100° C. and 40 mbar while stirring. The vacuum was then released, the mixture cooled to an internal temperature of 70-80° C., and the flask contents neutralized by adding 2.5 g of anhydrous soda (light sodium carbonate; CAS No. 497-19-8; available from Sigma-Aldrich, Munich, Germany). The mixture was stirred for a further 30 minutes and then filtered. This afforded a clear oil having a viscosity of ˜525 m Pas.

### Production of Amino Silicone Oil Emulsions E1-E3:

### Production of the Amino Silicone Oil Emulsion E1:

4.9 g of a 80% aqueous solution of isotridecyldecaethoxylate, commercially available under the trade name Lutensol TO 10 (from BASF) and 1.6 g of demineralized water are premixed at 4000 rpm using an Ultra-Turrax T 50 emulsifier (from Janke & Kunkel/IKA). 34.9 g of a hydroxy/methoxy-terminated copolymer composed of 3-(2-aminoethylamino)propylmethylsiloxy and dimethylsiloxy units and having an amine value of 0.14 meq/g and a viscosity of 4000 mm²/s (at 25° C.) is added in three portions at a shear rate of 4000 rpm, resulting in a relatively firm stiff phase as a pre-emulsion. This is diluted with 57.5 g of demineralized water added portionwise with low shear to afford the desired emulsion and mixed with 0.20 g of 80% acetic acid and 0.9 g of 2-phenoxyethanol.

This affords a smooth, low-viscosity white silicone oil emulsion E1 having a solids content of 39.8% and a pH of 5.0.

Measurement of the particle size distribution gives a D50 value of 160 nm.

### Production of the Amino Silicone Oil Emulsion E2:

30.0 g of a 80% aqueous solution of isotridecyldecaethoxylate, commercially available under the trade name Lutensol TO 10 (from BASF), 81.7 g of a trimethylsilyl-terminated copolymer composed of 3-(2-aminoethylamino)propyl-methylsiloxy and dimethylsiloxy units and having an amine value of 0.6 meq/g and a viscosity of 2500 mm²/s (at 25° C.), and 86.3 g of a trimethylsilyl-terminated polydimethylsiloxane having a viscosity of 60000 mm²/s (at 25° C.) are premixed at 500 rpm in the container of a laboratory planetary mixer (model Labmax, from Molteni). 39.6 g of demineralized water and 1.4 g of 80% acetic acid (at 500 rpm) are added and the mixture is homogenized in three cycles at a shear rate of 2500 rpm, resulting in a soft to medium-firm stiff phase as a pre-emulsion. This is diluted with 157.4 g of demineralized water added portionwise with relatively low shear to afford the desired emulsion and this is mixed with 3.6 g of 2-phenoxyethanol.

This affords a smooth, mobile white silicone oil emulsion E2 having a solids content of 48.9% and a pH of 5.5.

Measurement of the particle size distribution gives a D50 value of 180 nm.

### Production of the Amino Silicone Oil Emulsion E3:

32.0 g of a 80% aqueous solution of isotridecyldecaethoxylate, commercially available under the trade name Lutensol TO 10 (from BASF), 44.0 g of a hydroxy/methoxy-terminated copolymer composed of 3-(2-aminoethylamino)propyl-methylsiloxy and dimethylsiloxy units and having an amine value of 0.14 meq/g and a viscosity of 4000 mm²/s (at 25° C.), and 132.0 g of a trimethylsilyl-terminated polydimethylsiloxane having a viscosity of 60000 mm²/s (at 25° C.) are premixed at 500 rpm in the container of a laboratory planetary mixer (model Labmax, from Molteni). 20.0 g of demineralized water and 0.3 g of 80% acetic acid (at 500 rpm) are added and the mixture is homogenized in three cycles at a shear rate of 2500 rpm, resulting in a firm stiff phase as a pre-emulsion. This is diluted with 168.1 g of demineralized water added portionwise with relatively low shear to afford the desired emulsion and this is mixed with 3.6 g of 2-phenoxyethanol.

This affords a smooth, freely mobile white silicone oil emulsion E3 having a solids content of 51.3% and a pH of 5.0.

Measurement of the particle size distribution gives a D50 value of 257 nm.

### EXAMPLES 1-4

The following examples 1-4 represent production methods for the synthesis of inventive aqueous dispersions of precrosslinked organopolysiloxanes, which are used for producing inventive cosmetic compositions.

### EXAMPLE 1

### Emulsion B1-a=B1-b.

92.26 g of amino silicone oil emulsion E1 is homogenized with 7.74 g of oxalamido-ethyl-ester-terminated organopolysiloxane S1 at 5000 rpm within one minute using an Ultra-Turrax T 50 emulsifier (from Janke & Kunkel/IKA).

This affords a smooth, low-viscosity white silicone oil emulsion B1-a (=B1-b) having a solids content of 44.5% and a pH of 5.0. Measurement of the particle size distribution gives a D50 value of 141 nm.

After a drying time of 24 hours at 25° C., the emulsion is evaporated to afford a pronounced, whitish, slightly soft, elastic film that is slightly tacky on the surface.

### Emulsion B1-c.

96.16 g of amino silicone oil emulsion E1 is homogenized with 3.84 g of oxalamido-ethyl-ester-terminated organopolysiloxane S1 at 5000 rpm within one minute using an Ultra-Turrax T 50 emulsifier (from Janke & Kunkel/IKA).

This affords a smooth, low-viscosity white silicone oil emulsion B1-c having a solids content of 42.1% and a pH of 5.5. Measurement of the particle size distribution gives a D50 value of 123 nm.

After a drying time of 24 hours at 25° C., the emulsion is evaporated to afford a pronounced, whitish, slightly soft, elastic film.

### EXAMPLE 2

### Emulsion B2

96.08 g of amino silicone oil emulsion E2 is homogenized with 3.92 g of oxalamido-ethyl-ester-terminated organopolysiloxane S1 at 5000 rpm within one minute using an Ultra-Turrax T 50 emulsifier (from Janke & Kunkel/IKA).

This affords a smooth, mobile white silicone oil emulsion B2 having a solids content of 50.9% and a pH of 6.5. Measurement of the particle size distribution gives a D50 value of 230 nm.

After a drying time of 24 hours at 25° C., the emulsion is evaporated to afford a pronounced, whitish, slightly soft, elastic film.

### EXAMPLE 3

### Emulsion B3

98.22 g of amino silicone oil emulsion E3 is homogenized with 1.78 g of oxalamido-ethyl-ester-terminated organopolysiloxane S1 at 5000 rpm within one minute using an Ultra-Turrax T 50 emulsifier (from Janke & Kunkel/IKA).

This affords a smooth, freely mobile white silicone oil emulsion B3 having a solids content of 52.2% and a pH of 4.5. Measurement of the particle size distribution gives a D50 value of 210 nm.

After a drying time of 24 hours at 25° C., the emulsion is evaporated to afford a pronounced, whitish, slightly soft, elastic film that is slightly tacky on the surface.

### EXAMPLE 4

### Emulsion B4

85.69 g of amino silicone oil emulsion E1 is homogenized with 14.31 g of oxalamido-ethyl-ester-terminated organopolysiloxane S2 at 5000 rpm within one minute using an Ultra-Turrax T 50 emulsifier (from Janke & Kunkel/IKA).

This affords a smooth, low-viscosity white silicone oil emulsion B4 having a solids content of 48.4% and a pH of 5.5. Measurement of the particle size distribution gives a D50 value of 192 nm.

After a drying time of 24 hours at 25° C., the emulsion is evaporated to afford a pronounced, whitish, slightly soft, elastic film.

### Comparative Experiment V1:

The following comparative experiment V1 represents a production process for the synthesis of noninventive aqueous emulsions (crosslinking with noninventive diethyl oxalate in accordance with U.S. Pat. No. 5,039,738 A):

99.6 g of amino silicone oil emulsion E1 is homogenized with 0.4 g of diethyl oxalate at 5000 rpm within one minute using an Ultra-Turrax T 50 emulsifier (from Janke & Kunkel/IKA).

This affords a smooth, low-viscosity white silicone oil emulsion V1 having a solids content of 40.0% and a pH of 4.5. Measurement of the particle size distribution gives a D50 value of 71 nm.

After a drying time of 24 hours at 25° C., the emulsion is evaporated to obtain a white opaque, pasty layer that shows weak bonding to glass and aluminum.

### Comparative Experiment V2:

The following comparative experiment V2 represents a production process for the synthesis of noninventive aqueous emulsions (crosslinking with noninventive diethyl-L-tartrate in accordance with WO20239229 A):

99.4 g of amino silicone oil emulsion E1 is homogenized with 0.6 g of diethyl-L-tartrate at 5000 rpm within one minute using an Ultra-Turrax T 50 emulsifier (from Janke & Kunkel/IKA).

This affords a smooth, low-viscosity white silicone oil emulsion V2 having a solids content of 40.2% and a pH of 5.5. Measurement of the particle size distribution gives a D50 value of 135 nm.

After a drying time of 24 hours at 25° C., the emulsion is evaporated to afford a soft, milky-white, elastic film with a slightly tacky surface.

Rheology of the Elastomer Films after Removal of Water:

A Teflon ring having an internal diameter of 40 mm is placed onto a circular glass plate having a diameter of 65 mm, to form a casting mold. Into this ring, 2.5 g of the emulsion to be measured is weighed out onto the glass plate avoiding air bubbles. The cast emulsion is then allowed to dry at 25° C. and 101.425 kPa. It must be ensured that the casting mold with the cast emulsion is situated on a leveled flat surface while drying, so that a film of uniform thickness can develop. After drying, a film approx. 0.5 mm thick is obtained here.

After a standing time of 1 week at 25° C., the resulting films undergo rheological analysis. The measurements were carried out using an MCR 302 rheometer from Anton Paar, using a PP12.5 plate/plate measuring system and a gap height of 0.5 mm. The instrument was calibrated with standard oil 10000 from the Physikalisch-Technische Bundesanstalt [German Federal Physical-Technical Institute]. The measurement temperature is 25.00° C.±0.05° C.

The values listed in Table 1 for the storage modulus G′, the loss modulus G″ and the loss factor tan δ can be computed by applying a sinusoidal deformation to measure the shear stress T and the phase shift angle δ. The measured values listed in Table 1 were measured at a frequency of 1 Hz and a deformation of 0.1%. When undergoing deformation, the measured samples are in the linear viscoelastic measuring range.

The following relationship applies here: tan δ=G″/G′. If tan δ<1, the elastic character of the sample is predominant; if tan δ>1, the viscous character of the sample is predominant.

The results of the rheology measurements for the elastomer films of inventive examples B1 to B4, of comparative experiments V1 and V2 and also of the uncrosslinked amino silicone oil emulsions E1 to E3 are summarized in Table 1.

TABLE 1
Rheological data of the elastomer films

| | Storage modulus | Loss modulus | |
|---|---|---|---|
| Ex./Comp. | G′ [Pa] | G″ [Pa] | tan δ |
| B1-a = B1-b | 8409 | 4094 | 0.49 |
| B1-c | 13853 | 3872 | 0.28 |
| B2 | 3465 | 997 | 0.29 |
| B3 | 1881 | 1127 | 0.60 |
| B4 | 3386 | 1676 | 0.50 |
| V1 | 3182 | 3904 | 1.23 |
| V2 | 17196 | 5809 | 0.34 |
| E1 | 28 | 205 | 7.28 |
| E2 | 14 | 138 | 9.99 |
| E3 | 140 | 502 | 3.60 |

Table 1 demonstrates that the inventive emulsions B1 to B4, respectively comprising oxalamido-ethyl-ester-terminated organopolysiloxanes S1 and S2 as crosslinker component, form elastic films following removal of water, since tan δ<1.

The noninventive emulsion V1 does not show any elastic film formation following removal of water, since tan δ is greater than 1.

The noninventive emulsion V2 comprising diethyl-L-tartrate as crosslinker component shows the formation of an elastic film following removal of water in accordance with WO20239229 A1, since tan δ<1.

The evaporated, pure amino silicone oil emulsions E1 to E3 do not show film formation following removal of water, since tan δ is significantly greater than 1 in all cases. This shows that the addition of a crosslinker is necessary to achieve film formation.

### Test Methods for Assessing the Effect of Cosmetic Compositions:

### Natural Hair

The application behavior of the cosmetic composition and its effect in respect of combing force and softness were assessed on Caucasian hair, available from Kerling International Haarfabrik GmbH. Before being used, undamaged tresses of natural hair are cleaned and where appropriate, in a further process step, damaged by bleaching.

### Basic Cleaning

For cleaning, the undamaged hair tresses are placed in a solution of methyl isobutyl ketone for one hour and shaken. After removal of the solvent mixture, the hair tresses are washed in each case twice with 3 ml of ammonium lauryl sulfate solution (25%), STEPANOL(R) ALS 25, from STEPAN Company, and then with demineralized water having a temperature of 30° C. The tresses are detangled using a coarse-tooth comb. The hair tresses are then placed for an hour in a large beaker of demineralized water, taken out, and rinsed again under running demineralized water. After the basic cleaning, and before being used further, the tresses are conditioned for at least 12 hours at 23° C. and 50% humidity and are combed before being used.

### Bleaching of Hair —Producing Damaged Hair

Damaged hair is produced by bleaching cleaned natural hair tresses (from Kerling International Haarfabrik GmbH, Euro-natural hair, color 6/0, 20 cm, tress weight 2 g). This is done by placing five hair tresses in each case for 30 minutes into a solution composed of 30% hydrogen peroxide and 25% ammonia (ratio 33.5:1). The hair is then thoroughly rinsed with demineralized water and washed twice with 3 ml of ammonium lauryl sulfate solution (25%), STEPANOL(R) ALS 25, from STEPAN Company, and with demineralized water having a temperature of 30° C. The hair tresses are then placed for an hour in a large beaker of demineralized water, taken out, and rinsed again under running demineralized water. Before further treatment, the bleached tresses are conditioned for at least 12 hours at 23° C. and 50% humidity and are combed before being used.

### Combing Force Measurement:

The combing force of wet and dry hair was determined using bleached or unbleached hair tresses (from Kerling International Haarfabrik GmbH, Euro-natural hair, color 6/0, 20 cm, tress weight 2 g). The combing force was measured by the double comb method of Y. K. Kamath and Hans-Dietrich Weigmann, J. Soc. Cosmet. Chem., 37, 111-124, 1986, using an Instron 3343 tensile tester. First, the wet and dry combing force is determined along the measured section on untreated hair tresses. The hair tresses are then treated with an inventive cosmetic composition and the force absorption during the combing procedure determined. The measured value reported is the reduction in combing force along the measured section (work) that is obtained between the treated and untreated hair tress. The average value from five hair tresses is calculated. The combing force reduction is reported as a percentage.

### Suppleness/Softness (According to Tensile Testing):

The softness of the hair was determined using bleached or unbleached hair tresses (from Kerling International Haarfabrik GmbH, Euro-natural hair, color 6/0, 20 cm, tress weight 2 g). The hair softness in the dry state was determined using an Instron 3343 tensile tester, by correlating the required tensile force with the parameters flexural stiffness and surface roughness of the hair bundle. These two parameters correlate in turn with the hair softness. For this purpose, an untreated hair tress was clamped into a measurement assembly consisting of five rods positioned opposite one another at an offset. The shape of the hair tress in this starting position is a kind of double S. Following this preparation, the hair tress is pulled out of the measurement assembly in one direction and the force required is evaluated along the measured section, as work. The hair tresses are then treated with an inventive cosmetic composition and the force absorption on pulling of the hair tress through the measurement assembly is determined along the measured section. The measured value reported is the reduction in tensile force along the measured section (work) that is obtained between the treated and untreated hair tress. A large reduction in tensile force (work) corresponds to a good soft feel/high suppleness. The average value from five hair tresses is calculated.

### Softness (According to Panel Test):

For assessing the softness of hair tresses, their tactile properties are assessed by experts (trained panelists). The hair tresses are compared in pairs in each case—for example, shampoo-treated hair as compared with untreated hair. The number of pairs of tresses assessed is at least three, the number of panelists at least five. The evaluation was based on hair tresses from Kerling International Haarfabrik GmbH (Euro-natural hair, color 6/0, 20 cm, tress weight 2 g).

### Washing Procedure, Shampoo:

0.2 g of shampoo per g of hair is applied to a cleaned, wetted hair tress. The shampoo is massaged in for 30 seconds in the direction of the hair ends. The hair tress is then rinsed for 30 s under running demineralized water and detangled using a coarse-tooth comb. The procedure is repeated twice. On the last occasion, the rinsing process is extended to 60 s. The hair tress is then dried for at least 12 h at a humidity of 50% and a temperature of 23° C.

### Washing Procedure, Conditioner:

0.3 g of rinse-off conditioner per g of hair is applied to a cleaned, wetted hair tress. The rinse-off conditioner is massaged in for 120 seconds in the direction of the hair ends. The hair tress is then rinsed for 60 s under running demineralized water and detangled using a coarse-tooth comb. The procedure is repeated. The hair tress is then dried for at least 12 h at a humidity of 50% and a temperature of 23° C.

### Determination of the Amount of Si Deposited on the Hair Surface in Ppm (Silicone Deposition):

The amount of silicone deposited on the hair surface is determined using an energy-dispersive x-ray fluorescence spectrometer (AMETEK, XEPOS). The hair bundles are placed in a specially produced sample holder having a circular measurement area 12 mm in diameter. The hair surface in the region of the measurement area is smooth, and the hairs are aligned in parallel. The sample is excited under the helium atmosphere, using a palladium tube (17.05 kV, 2.0 mA). The excitation time is 300 s. Control samples (natural hair tresses) are subjected to regular measurement. In the event of deviations, a drift correction is carried out using glass tablets. The calibration standards used were hair tresses loaded with polydimethylsiloxane in the 50 to 2000 ppm range (control by atomic absorption spectroscopy).

For determination of the effectiveness of the silicone deposition, the amount of Si in ppm on a cleaned hair bundle is first determined=blank value. The same hair bundle is then treated, for example by being washed with a shampoo. The amount of Si in ppm is determined again=sample value. The amount of Si in ppm deposited is obtained by performing the following subtraction: sample value—blank value. Each hair tress is measured centrally on the facing and reverse sides. The result reported is the average value from three hair tresses.

### Simulation of Shampooing by Stirring Hair Tresses in a Surfactant Solution:

Many users want cosmetic effects such as improved hair softness, reduction in wet combing force, and retention of hair color to persist following use of a hair-conditioning product, in spite of multiple subsequent washes with shampoo. To assess the persistence of cosmetic effects following treatment of hair with an inventive cosmetic composition, a method was developed that constitutes a simulation of successive shampooings. For this purpose a treated hair tress in a 100 ml jar with screw-top lid is treated with 50 ml of a five percent solution of ammonium lauryl sulfate, this solution having been thermally equilibrated at 40° C. and obtained by diluting STEPANOL® ALS 25, from STEPAN Company, and the treated tress is shaken for a defined time in an incubation shaker (from Heidolph Unimax 1010+Incubator 1000) thermally equilibrated at 40° C., at a speed of 250 rpm. After having been shaken, the tresses are rinsed for one minute with demineralized water having a temperature of 30° C. and dried.

### Curl Retention Test at High Humidity:

The curl retention test at high humidity allows the assessment of care and styling products in respect of shaping properties for hair fibers. In this model for determining hair fixing, the percentage changes from the initial to final length of locks of hair curled in a defined manner are recorded versus hair tress length after. The curl retention properties of hair care and styling products at high humidity are monitored at 23° C. and 90% relative humidity over a period of seven hours.

Hair tresses of brown European hair 15 cm in length are assembled into bundles of 3.5 g, bound with thread, and permanently fixed with a suitable adhesive. The hair tresses are washed in each case twice with 3 ml of ammonium lauryl sulfate solution (25%), STEPANOL(R) ALS 25, from STEPAN Company, and then with demineralized water having a temperature of 30° C. The hair tresses are combed and dried. The dry tresses are sprayed with 20 puffs of a 3% active pump spray, combed once, and then wound around a plastic rod having a diameter of 1.4 cm, temporarily fixed with a cover, and dried overnight at 50° C.

The curled locks of hair are carefully removed from the plastic rods. After cooling briefly, the curled locks of hair are attached to a scaled hanging device in a climatic chamber at 23° C. and 90% relative humidity. The initial length of the curled lock of hair had previously been determined and recorded. At defined time intervals, the lengths of the curled locks of hair, i.e. the change from the initial length, are read over a period of 7 h.

The values are calculated according to equation (G-I),

$$\% CURL RETENTION = \frac{L - {L}_{t}}{L - {L}_{0}} \times 100 \qquad ( G - I )$$

where
L=Length of the hair tress
Lₒ=Initial length of the curled lock of hair
L_{f}=Length of the curled lock of hair after/during the measurement

The higher the curl retention percentage after a time t, the better the shaping properties/fixing properties of the cosmetic formulation. The uncurling of the curled locks of hair is less pronounced.

### EXAMPLES OF COSMETIC COMPOSITIONS

Precrosslinked emulsions are preferably emulsions that, after removal of water, form elastic films and for which tan δ<1 in rheological measurements.

### EXAMPLES A1-A, A1-C, AND A2-A (RINSE-OFF CONDITIONER)

The following examples represent the inventive cosmetic compositions A1-a, A1-c, and A2-a shown in Table 2, comprising precrosslinked emulsions B1-a, B1-c, and B2 from the corresponding examples. The active content of organopolysiloxanes in the cosmetic compositions is 0.5%.

### Preparation Instructions:

An initial charge of water is heated to 75° C. with stirring. 2.0 parts of hydroxyethyl cellulose are added. On reaching 65° C., 0.5 parts of Polysorbate 80, 0.5 parts of Stearyl Alcohol, 0.5 parts of Cetyl Alcohol, and 0.2 parts of Behentrimonium Chloride are added. The mixture is stirred until 75° C. is reached and the ingredients are in solution. The mixture is then cooled. In the course of cooling, 0.1 parts of Citric Acid and 0.2 parts of Tetrasodium EDTA are added. At 35° C., 0.9 parts of Phenoxyethanol, and Ethylhexylglycerin are added. Stirring is continued and the emulsion from the examples is added. The composition is homogenized with stirring for 15 minutes.

TABLE 2
Rinse-off conditioner A1-a, A1-c, and A2-a

| | Ex. | Ex. | Ex. |
|---|---|---|---|
| | A1-a | A1-c | A2-a |
| Constituents | [parts by | [parts by | [parts by |
| (INCI name) | weight] | weight] | weight] |
| Water | to 100 | to 100 | to 100 |
| Hydroxyethylcellulose¹⁾ | 2.0 | 2.0 | 2.0 |
| Cetyl Alcohol ²⁾ | 0.5 | 0.5 | 0.5 |
| Polysorbate 80 ³⁾ | 0.5 | 0.5 | 0.5 |
| Behentrimonium Chloride ⁴⁾ | 0.2 | 0.2 | 0.2 |
| Stearyl Alcohol ⁵⁾ | 0.5 | 0.5 | 0.5 |
| Citric Acid ⁶⁾ | 0.1 | 0.1 | 0.1 |
| Tetrasodium EDTA ⁷⁾ | 0.2 | 0.2 | 0.2 |
| Emulsion B1-a | 1.43 | | |
| from example 1 | | | |
| Emulsion B1-c | | 1.43 | |
| from example 1 | | | |
| Emulsion B2 | | | 1.16 |
| from Example 2 | | | |
| Phenoxyethanol, | 0.9 | 0.9 | 0.9 |
| Ethylhexylglycerin ⁸⁾ | | | |

The raw materials listed in Table 2 are available under the following trade names:
¹⁾Hydroxyethylcellulose: Natrosol 250 HR, Ashland
²⁾ Cetyl Alcohol: Cetyl alcohol, Merck KGaA
³⁾ Polysorbate 80: Tween ™ 80, Croda GmbH
⁴⁾ Behentrimonium Chloride: Genamin ® KDMP, Clariant GmbH
⁵⁾ Stearyl Alcohol: Stearyl alcohol Merck KGaA
⁶⁾ Citric Acid: Citric acid, Sigma
⁷⁾ Tetrasodium EDTA: EDETA ® B powder, BASF Corporation
⁸⁾ Phenoxyethanol, Ethylhexylglycerin: Euxyl PE 9010, Schülke & Mayr

### Comparative Experiments VA1 and VA2 (Rinse-Off Conditioner (Rinse))

The following comparative experiments VA1 and VA2 represent noninventive cosmetic compositions comprising the non-precrosslinked aqueous dispersions E1 and E2 from the corresponding examples. The active content of organopolysiloxanes in the cosmetic compositions is 0.5%.

For the preparation of the cosmetic compositions of comparative experiments VA1 and VA2, the procedure for examples A1-a, A1-c, and A2-a was repeated, with the modification that instead of the emulsions B1-a, B1-c, and B2 (emulsions of inventive precrosslinked organopolysiloxanes) the uncrosslinked emulsions E1 and E2 are used.

### Preparation Instructions:

An initial charge of water is heated to 75° C. with stirring. 2.0 parts of hydroxyethyl cellulose are added. On reaching 65° C., 0.5 parts of Polysorbate 80, 0.5 parts of Stearyl Alcohol, 0.5 parts of Cetyl Alcohol, and 0.2 parts of Behentrimonium Chloride are added. The mixture is stirred until 75° C. is reached and the ingredients are in solution. The mixture is then cooled. In the course of cooling, 0.1 parts of Citric Acid and 0.2 parts of Tetrasodium EDTA are added. At 35° C., 0.9 parts of Phenoxyethanol, and Ethylhexylglycerin are added. Stirring is continued and the emulsion shown in Table 3 for the comparative experiments is added. The composition is homogenized with stirring for 15 minutes.

TABLE 3
Rinse-off conditioners VA1 and VA2

| Constituents | See exp. VA1 | See exp. VA2 |
|---|---|---|
| (INCI name) | [parts by weight] | [parts by weight] |
| Water | to 100 | to 100 |
| Hydroxyethylcellulose ¹⁾ | 2.0 | 2.0 |
| Cetyl Alcohol ²⁾ | 0.5 | 0.5 |
| Polysorbate 80 ³⁾ | 0.5 | 0.5 |
| Behentrimonium | 0.2 | 0.2 |
| Chloride ⁴⁾ | | |
| Stearyl Alcohol ⁵⁾ | 0.5 | 0.5 |
| Citric Acid ⁶⁾ | 0.1 | 0.1 |
| Tetrasodium EDTA ⁷⁾ | 0.2 | 0.2 |
| Emulsion E1 | 1.43 | |
| Emulsion E2 | | 1.16 |
| Phenoxyethanol, | 0.9 | 0.9 |
| Ethylhexylglycerin⁸⁾ | | |

The raw materials listed in Table 3 are available under the following trade names:
¹⁾ Hydroxyethylcellulose: Natrosol 250 HR, Ashland
²⁾ Cetyl Alcohol: Cetyl alcohol, Merck KGaA
³⁾ Polysorbate 80: Tween ™ 80, Croda GmbH
⁴⁾ Behentrimonium Chloride: Genamin ® KDMP, Clariant GmbH
⁵⁾ Stearyl Alcohol: Stearyl alcohol Merck KGaA
⁶⁾ Citric Acid: Citric acid, Sigma
⁷⁾ Tetrasodium EDTA: EDETA ® B powder, BASF Corporation
⁸⁾Phenoxyethanol, Ethylhexylglycerin: Euxyl PE 9010, Schülke & Mayr

### Comparative Experiment VA1-DEO (Rinse-Off Conditioner (Rinse))

The following comparative experiment VA1-DEO represents a noninventive cosmetic composition comprising an aqueous dispersion of a precrosslinked organopolysiloxane. The active content of organopolysiloxanes in the cosmetic composition is 0.5%.

For the preparation of the cosmetic composition of comparative experiment VA1-DEO, the procedure for examples VA1 and VA2 was repeated, with the modification that instead of the emulsions E1 and E2 (emulsions of non-precrosslinked organopolysiloxanes) the precrosslinked, noninventive emulsion V1 is used.

### Preparation Instructions:

An initial charge of water is heated to 75° C. with stirring. 2.0 parts of hydroxyethyl cellulose are added. On reaching 65° C., 0.5 parts of Polysorbate 80, 0.5 parts of Stearyl Alcohol, 0.5 parts of Cetyl Alcohol, and 0.2 parts of Behentrimonium Chloride are added. The mixture is stirred until 75° C. is reached and the ingredients are in solution. The mixture is then cooled. In the course of cooling, 0.1 parts of Citric Acid and 0.2 parts of Tetrasodium EDTA are added. At 35° C., 0.9 parts of Phenoxyethanol, and Ethylhexylglycerin are added. Stirring is continued and the emulsion shown in Table 4 for the comparative experiment is added. The composition is homogenized with stirring for 15 minutes.

TABLE 4
Rinse-off conditioner VA1-DEO

| | See exp. |
|---|---|
| Constituents | VA1-DEO |
| (INCI name) | [parts by weight] |
| Water | to 100 |
| Hydroxyethylcellulose ¹⁾ | 2.0 |
| Cetyl Alcohol ²⁾ | 0.5 |
| Polysorbate 80 ³⁾ | 0.5 |
| Behentrimonium | 0.2 |
| Chloride ⁴⁾ | |
| Stearyl Alcohol ⁵⁾ | 0.5 |
| Citric Acid ⁶⁾ | 0.1 |
| Tetrasodium EDTA ⁷⁾ | 0.2 |
| Emulsion V1 | 1.43 |
| Phenoxyethanol, | 0.9 |
| Ethylhexylglycerin⁸⁾ | |

The raw materials listed in Table 4 are available under the following trade names:
¹⁾ Hydroxyethylcellulose: Natrosol 250 HR, Ashland
²⁾ Cetyl Alcohol: Cetyl alcohol, Merck KGaA
³⁾ Polysorbate 80: Tween ™ 80, Croda GmbH
⁴⁾ Behentrimonium Chloride: Genamin ® KDMP, Clariant GmbH
⁵⁾ Stearyl Alcohol: Stearyl alcohol Merck KGaA
⁶⁾ Citric Acid: Citric acid, Sigma
⁷⁾ Tetrasodium EDTA: EDETA ® B powder, BASF Corporation
⁸⁾Phenoxyethanol, Ethylhexylglycerin: Euxyl PE 9010, Schülke & Mayr

Comparison of the Rinse-Off Conditioners of Inventive Examples A1-a, A1-c, and A2-a with Comparative Experiments VA1 and VA2

The recited examples and comparative experiments differ in that, in the case of examples A1-a, A1-c, and A2-a, inventive aqueous dispersions of precrosslinked organopolysiloxanes were used, whereas the comparative experiments VA1 and VA2 used in each case analogous aqueous dispersions of the corresponding non-precrosslinked organopolysiloxanes. The active content of organopolysiloxanes in the cosmetic compositions is 0.5%.

In the direct comparison, the following examples/comparative experiments correlate:
Example A1-a—Comparative experiment VA1
Example A1-c—Comparative experiment VA1
Example A2-a—Comparative experiment VA2
   Wet Combing Force after Treatment of Damaged Hair with Rinse-Off Conditioner and after Simulation of Multiple Shampooings by Stirring the Hair in an Aqueous Surfactant Solution (Persistence Effect)

### EXAMPLES A1-A AND A1-C —COMPARATIVE EXPERIMENT VA1

The use of the aqueous dispersion B1-a and B1-c of inventively precrosslinked organopolysiloxanes in the rinse-off conditioner (examples A1-a and A1-c) leads to an improvement in conditioning properties, such as a reduction in the combing forces in the wet state by comparison with the noninventive rinse-off conditioner from example VA1, which comprises the dispersion of a non-precrosslinked organopolysiloxane E1. An additional aim of the rinse-off conditioners of the invention is that the caring properties are retained even after multiple shampooing of the hair. The shampooing process is in this example simulated by the stirring of hair bundles, treated with rinse-off conditioner, for four hours in a surfactant solution. The details of this treatment are described above under Test methods.

The results of the determination of the combing force in the wet state are set out below for the rinse-off conditioners of examples A1-a, A1-c, and VA1 in Table 5.

TABLE 5
Rinse-off conditioners/results for reduction in wet combing force
on damaged Caucasian hair after treatment with an inventive rinse-
off conditioner by comparison with a noninventive conditioner
and after simulation of multiple shampooings by four-hour stirring
of the hair in an aqueous surfactant solution. All results refer
to the comparison relative to untreated hair tresses.

| | | Reduction in wet |
|---|---|---|
| | | combing force after |
| | | conditioner treatment and |
| | | subsequent stirring |
| | Reduction in wet combing | of the hair in an |
| | force after conditioner | aqueous surfactant solution |
| Ex./ | treatment | (persistence test) |
| Comp. | [%] | [%] |
| A1-a | 84 | 39 |
| A1-c | 81 | 31 |
| VA1 | 79 | 21 |

Through treatment with the inventive rinse-off conditioner comprising an aqueous emulsion of a precrosslinked organopolysiloxane B1-a (example A1-a), with a value of 84% it is possible to achieve a significant reduction in the measured wet combing force of the hair tresses. In particular, a high conditioning effect is retained after stirring the treated hair in a surfactant solution, and this is reflected in a persistent reduction in wet combing force of 39%. Similarly, treatment with an inventive rinse-off conditioner comprising the aqueous emulsion of a precrosslinked organopolysiloxane B1-c (example A1-c) achieves a high measured reduction of 81% in the wet combing force of the hair tresses. After stirring the treated hair in surfactant solution, a reduction in wet combing force of 31% persists.

Hair treated with a noninventive rinse-off conditioner comprising the emulsion E1 (comparative example VA1) exhibits a slightly decreased reduction in wet combing force, of 79%. The reduction in wet combing force of 21% following the surfactant treatment is however significantly lower by comparison with example A-1a and A-1c. The treatment of the hair in surfactant solution represents a simulation of multiple hair washes with shampoo, and demonstrates that the inventive rinse-off conditioners exhibit better wash resistance and that the conditioning properties are retained for longer than in the case of hair treatment with a noninventive rinse-off conditioner.

Comparison of Silicone Deposition after Treatment of Damaged Hair with Rinse-Off Conditioner and after Simulation of Multiple Shampooings by Four-Hour Stirring of the Hair in an Aqueous Surfactant Solution

### EXAMPLE A2-A—COMPARATIVE EXPERIMENT VA2

As a further criterion, the efficiency of silicone deposition on damaged hair after treatment with rinse-off conditioners was evaluated, in addition to which the amount of the conditioning organopolysiloxane that remains on the hair surface after simulation of shampooings by four-hour stirring of the hair in a surfactant solution was investigated. The results are summarized in Table 6. The determination of silicone deposition is described above under Test methods.

TABLE 6
Rinse-off conditioners/silicone deposition

| | | Silicone deposition after stirring the |
|---|---|---|
| | | hair in an aqueous surfactant |
| Ex./ | Silicone deposition | solution (persistence test) |
| Comp. | [ppm] | [ppm] |
| A2-a | 55 | 34 |
| VA2 | 36 | 21 |

Table 6 shows that the deposition of silicone—even after shampooing—in the inventive example A2-a is both more effective and more resistant to shampooing by comparison with the noninventive comparative experiment VA2.

Comparison of the Rinse-Off Conditioners of Inventive Example A1-a with the Comparative Experiment VA1-DEO

The recited example and the comparative experiment differ in that, in the case of example A1-a, an inventive aqueous dispersion of precrosslinked organopolysiloxanes was used, whereas comparative experiment VA1-DEO used analogous noninventive aqueous dispersions of the corresponding precrosslinked organopolysiloxanes. The active content of organopolysiloxanes in the cosmetic compositions is 0.5%.

Wet Combing Force after Treatment of Damaged Hair with Rinse-Off Conditioner and after Simulation of Multiple Shampooings by Stirring the Hair in an Aqueous Surfactant Solution (Persistence Effect)

### EXAMPLE A1-A—COMPARATIVE EXPERIMENT VA1-DEO

The use of the aqueous dispersion B1-a of inventively precrosslinked organopolysiloxanes in the rinse-off conditioner (example A1-a) leads to an improvement in conditioning properties, such as a reduction in the combing forces in the wet state by comparison with the noninventive rinse-off conditioner from example VA1-DEO, which comprises the dispersion of a precrosslinked organopolysiloxane (comparative experiment V1). An additional aim of the rinse-off conditioner of the invention is that the caring properties are retained even after multiple shampooing of the hair. The shampooing process is in this example simulated by the stirring of hair bundles, treated with rinse-off conditioner, for four hours in a surfactant solution. The details of this treatment are described above under Test methods.

The results of the determination of the combing force in the wet state are set out below for the rinse-off conditioners of examples A1-a and VA1-DEO in Table 7.

TABLE 7
Rinse-off conditioners/results for reduction in wet combing force
on damaged Caucasian hair after treatment with an inventive rinse-
off conditioner by comparison with a noninventive conditioner
and after simulation of multiple shampooings by four-hour stirring
of the hair in an aqueous surfactant solution. All results refer
to the comparison relative to untreated hair tresses.

| | | Reduction in wet |
|---|---|---|
| | | combing force after |
| | | conditioner treatment and |
| | | subsequent stirring |
| | Reduction in wet combing | of the hair in an |
| | force after conditioner | aqueous surfactant solution |
| Ex./ | treatment | (persistence test) |
| Comp. | [%] | [%] |
| A1-a | 84 | 39 |
| VA1-DEO | 77 | 20 |

Through treatment with the inventive rinse-off conditioner comprising an aqueous emulsion of a precrosslinked organopolysiloxane B1-a (example A1-a), with a value of 84% it is possible to achieve a significant reduction in the measured wet combing force of the hair tresses. In particular, a high conditioning effect is retained after stirring the treated hair in a surfactant solution, and this is reflected in a persistent reduction in wet combing force of 39%.

Hair treated with a noninventive rinse-off conditioner VA1-DEO comprising the emulsion from comparative example V1 exhibits a slightly reduced reduction in wet combing force, of 77%. The reduction in wet combing force of 20% following the surfactant treatment is however significantly lower by comparison with example A-1a. The treatment of the hair in surfactant solution represents a simulation of multiple hair washes with shampoo, and demonstrates that the inventive rinse-off conditioner exhibits better wash resistance and that the conditioning properties are retained for longer than in the case of hair treatment with a noninventive rinse-off conditioner.

### EXAMPLE A3-A

### Cosmetic Composition: Shampoo

The following example represents an inventive cosmetic composition A3-a comprising the emulsion B3. The comparative example VA3 represents a commercially available shampoo formulation comprising an emulsion of a dimethicone (dimethylpolysiloxane; BELSIL® DM 5102 E, available from Wacker Chemie AG) having a viscosity of 60 000 mm²/s (at 25° C.). The active content of organopolysiloxanes in the cosmetic compositions is 1.3%.

The composition of the shampoos is summarized in Table 8.

### Preparation Instructions:

An initial charge of 32.11 parts of water is heated to 50° C. with stirring. During this time, 0.20 parts of Guar Hydroxypropyltrimonium Chloride, 6.06 parts of Sodium Lauryl Sulfate, 29.90 parts of Sodium Laureth Sulfate, 0.05 parts of Citric Acid, and 5.0 parts of Cocamidopropyl Betaine are added. The mixture is stirred until 50° C. is reached and the ingredients are in solution. The mixture is then cooled. A separate vessel is charged with 20.0 parts of water, 0.60 parts of Carbomer are added with stirring, and stirring is continued until a homogeneous mixture is obtained. 0.06 parts of Lactic Acid are then added. This mixture is added to the first mixture. At 40° C., 0.95 parts of Phenoxyethanol, and Ethylhexylglycerin are added. Stirring is continued and 0.30 parts of C12-13 Alkyl Lactate, 2.91 parts of inventive emulsion B3 or 2.60 parts of the noninventive Dimethicone Emulsion BELSIL® DM 5102 E, 0.40 parts of Sodium Hydroxide, and 0.66 parts of Sodium Chloride are added. The pH can be adjusted to the required value of 6.5 by addition of sodium hydroxide as necessary.

TABLE 8
Shampoo formulation A3-a and VA3

| | | Comparative experiment |
|---|---|---|
| Constituents | Example A3-a | VA3 |
| (INCI Name) | [parts by weight] | [parts by weight] |
| Citric Acid¹⁾ | 0.05 | 0.05 |
| Cocamidopropyl Betaine²⁾ | 5.00 | 5.00 |
| Sodium Laureth Sulfate³⁾ | 29.90 | 29.90 |
| Guar Hydroxypropyl- | 0.20 | 0.20 |
| trimonium Chloride⁴⁾ | | |
| Sodium Lauryl Sulfate⁵⁾ | 6.06 | 6.06 |
| Aqua (DI water) | 32.11 | 32.11 |
| Carbomer⁶⁾ | 0.60 | 0.60 |
| Lactic Acid⁷⁾ | 0.06 | 0.06 |
| Aqua (DI water) | 20.00 | 20.00 |
| Phenoxyethanol, | 0.95 | 0.95 |
| Ethylhexylglycerin⁸⁾ | | |
| C12-13 Alkyl Lactate⁹⁾ | 0.30 | 0.30 |
| Emulsion B3 | 2.91 | |
| from example 3 | | |
| BELSIL ® DM 5102 E¹⁰⁾ | | 2.60 |
| (dimethicone emulsion) | | |
| Sodium Hydroxide¹¹⁾ | 0.40 | 0.40 |
| Sodium Chloride¹²⁾ | 0.66 | 0.66 |

¹⁾Citric Acid: Citric acid, Sigma
²⁾Cocamidopropyl Betaine: Genagen CAB 818 30%, Clariant
³⁾Sodium Laureth Sulfate: Genapol ® LRO 26.5%, Clariant
³⁾Glycol Distearate: Genapol ® PMS, Clariant GmbH
⁴⁾Guar Hydroxypropyltrimonium Chloride: N-Hance ® BF 13, Ashland
⁵⁾Sodium Lauryl Sulfate: Texapon K 12 G, BASF
⁶⁾Carbomer: Carbopol 980, Lubrizol
⁷⁾Lactic acid: L-(+)Lactic acid, 90%, Bernd Kraft GmbH
⁸⁾Phenoxyethanol, Ethylhexylglycerin: Euxyl PE 9010, Schülke & Mayr
⁹⁾C12-13 Alkyl Lactate: Ceraphyl ™ 41 ester, Ashland
¹⁰⁾BELSIL ® DM 5102 E, available from Wacker Chemie AG
¹¹⁾Sodium Hydroxide: Sodium hydroxide, Sigma-Aldrich
¹²⁾Sodium Chloride: Sodium chloride pure, Bernd Kraft GmbH
Comparison of the shampoos of inventive example A3-a with the comparative experiment VA3

The recited example and the comparative experiment differ in that, in the case of example A-3a, an aqueous dispersion of precrosslinked organopolysiloxanes was used. In comparative experiment VA3, the commercial aqueous dimethylpolysiloxane emulsion BELSIL® DM 5102 E (Wacker Chemie AG) is used, which as active conditioning ingredient comprises a dimethicone (dimethylpolysiloxane) having a viscosity of 60 000 mm²/s. The active content of organopolysiloxanes in the cosmetic compositions is 1.3%.

The results for the cosmetic effect of the shampoos are summarized in Table 9.

TABLE 9
Shampoo/results for reduction in wet combing force and improvement
in softness on damaged hair after treatment with shampoo. All
results are by comparison with untreated hair tresses.

| | Reduction in wet | Improvement |
|---|---|---|
| | combing force | in softness |
| Ex./Comp. | [%] | [%] |
| Ex. A3-a | 50 | 25 |
| Comp. ex. | 20 | 2 |
| VA3*) | | |

*)BELSIL ® DM 5102 E (dimethicone emulsion), available from Wacker Chemie AG

The shampoos of example A-3a with the inventive emulsion exhibit a substantially higher reduction in wet combing force and significantly improved softness (according to tensile testing), by comparison with the shampoo of comparative experiment VA3 with the commercial dimethicone emulsion.

### EXAMPLE A3-B

### Cosmetic Composition—Shampoo

The following example represents a cosmetic composition comprising the emulsion B3.

The active content of organopolysiloxanes in the cosmetic composition is 1.0%.

The composition of the shampoo is summarized in Table 10.

### Preparation Instructions:

0.30 parts of Guar Hydroxypropyltrimonium Chloride are dispersed in water. 41.50 parts of Sodium Laureth Sulfate are stirred in slowly and the mixture is gradually heated to 75° C. In the course of the heating, 0.20 parts of PEG-150 Distearate are added at 50° C. and 0.50 parts of Glycol Distearate at 65° C. The mixture is then cooled. On reaching 35° C., 0.90 parts of Phenoxyethanol, Ethylhexylglycerin, and the emulsion corresponding to the example are added and the mixture is stirred for 5 minutes. Lastly, 13.4 parts of Cocamidopropyl Betaine are added and the mixture is stirred for a further 10 minutes.

Damaged hairs treated with the shampoo from example A3-b are according to the panel test softer than damaged, untreated hairs.

TABLE 10
Shampoo formulations A3-b (amounts in parts by weight)

| Constituents | |
|---|---|
| (INCI name) | Ex. A3-b |
| Aqua (DM water) | to 100 |
| Guar Hydroxypropyltrimonium Chloride ¹⁾ | 0.30 |
| Sodium Laureth Sulfate ²⁾ | 41.50 |
| Glycol Distearate ³⁾ | 0.50 |
| PEG-120 Methyl Glucose Dioleate 4) | 0.20 |
| Emulsion B3 from example 3 | 2.27 |
| Cocamidopropyl Betaine ⁶⁾ | 13.33 |
| Phenoxyethanol, Ethylhexylglycerin ⁷⁾ | 0.90 |

¹⁾ Guar Hydroxypropyltrimonium Chloride: N-Hance ® 3196, Ashland.
²⁾ Sodium Laureth Sulfate: Genapol ® LRO 26.5%, Clariant GmbH
³⁾ Glycol Distearate: HALLSTAR ® EGDS, The Hallstar Company
4) PEG-120 Methyl Glucose Dioleate: Glucamate ™ DOE 120 thickener, Lubrizol
5) BELSIL ® DM 5102 E, available from Wacker Chemie AG
⁶⁾ Cocamidopropyl Betaine: Genagen ® CAB 30%, Clariant GmbH
⁷⁾ Phenoxyethanol, Ethylhexylglycerin: Euxyl PE 9010, Schülke & Mayr

### EXAMPLE A3-C

### Cosmetic Composition—Shampoo

The following example represents a cosmetic composition comprising the emulsion B3.

The active content of organopolysiloxanes in the cosmetic composition is 1.0%.

The composition of the shampoo is summarized in Table 11.

TABLE 11
Shampoo formulation A3-c (amounts in parts by weight)

| Constituents | |
|---|---|
| (INCI name) | Ex. A3-c |
| Aqua (DM water) | to 100 |
| Polyquaternium-10 ¹⁾ | 0.10 |
| Sodium Laureth Sulfate ²⁾ | 52.80 |
| PEG-150 Distearate ³⁾ | 0.25 |
| Cocamide MEA ⁴⁾ | 1.00 |
| Emulsion B3 from example 3 | 2.27 |
| Cocamidopropyl Betaine ⁵⁾ | 10.06 |
| Phenoxyethanol, Ethylhexylglycerin ⁶⁾ | 0.95 |

¹⁾ Polyquaternium-10: UCARE Polymer JR 400, Dow Chemical.
²⁾ Sodium Laureth Sulfate: Genapol ® LRO 26.5%, Clariant GmbH
³⁾ PEG-150 Distearate: Eumulgin ® EO 33, BASF AG
⁴⁾ Cocamide MEA: Comperlan 100, BASF AG
⁵⁾ Cocamidopropyl Betaine: Genagen ® CAB 30%, Clariant GmbH
7) Phenoxyethanol, Ethylhexylglycerin: Euxyl PE 9010, Schülke & Mayr

Damaged hairs treated with the shampoo from example A3-c are according to the panel test softer than damaged, untreated hairs.

### EXAMPLES A1-B AND A2-B

### Rinse-Off Conditioner

The following examples represent cosmetic compositions A1-b and A2-b comprising the emulsions B-1b and B2. The active content of organopolysiloxanes in the cosmetic compositions is 2.0%.

The composition of the rinse-off conditioners is summarized in Table 12.

### Preparation Instructions:

An initial charge of water is heated to 75° C. with stirring. 1.1 parts of hydroxyethyl cellulose are added. On reaching 65° C., 0.5 parts of Stearamidopropyl Dimethylamine, 1.0 parts of Polysorbate 80, 3.0 parts of Stearyl Alcohol, 2.0 parts of Cetyl Alcohol, and 1.8 parts of Behentrimonium Chloride are added. The mixture is stirred until 75° C. is reached and the ingredients are in solution. The mixture is then cooled. In the course of cooling, 0.2 parts of Citric Acid and 0.2 parts of Tetrasodium EDTA are added. At 35° C., 0.9 parts of Phenoxyethanol, and Ethylhexylglycerin are added. Stirring is continued and the emulsions from the examples are added. The composition is homogenized with stirring for 15 minutes.

TABLE 12
Rinse-off conditioners A1-b and A2-b

| | Example A1-b | Example A2-b |
|---|---|---|
| Constituents | [parts | [parts |
| (INCI name) | by weight] | by weight] |
| Water | to 100 | to 100 |
| Hydroxyethylcellulose ¹⁾ | 1.1 | 1.1 |
| Cetyl Alcohol ²⁾ | 2.0 | 2.0 |
| Polysorbate 80 ³⁾ | 1.0 | 1.0 |
| Behentrimonium Chloride ⁴⁾ | 1.8 | 1.8 |
| Stearamidopropyl Dimethylamine ⁵⁾ | 0.5 | 0.5 |
| Stearyl Alcohol ⁶⁾ | 3.0 | 3.0 |
| Citric Acid ⁷⁾ | 0.2 | 0.2 |
| Tetrasodium EDTA ⁸⁾ | 0.2 | 0.2 |
| Emulsion B1-b from example 1 | 5.71 | |
| Emulsion B2 from example 2 | | 4.64 |
| Phenoxyethanol, | 0.9 | 0.9 |
| Ethylhexylglycerin ⁹⁾ | | |

The raw materials listed in Table 12 are available under the following trade names:
¹⁾ Hydroxyethylcellulose: Natrosol 250 HR, Ashland
²⁾ Cetyl Alcohol: Cetyl alcohol, Merck KGaA
³⁾ Polysorbate 80: Tween ™ 80, Croda GmbH
⁴⁾ Behentrimonium Chloride: Genamin ® KDMP, Clariant GmbH
⁵⁾ Stearamidopropyl Dimethylamine, Incromine ™ SB, Croda GmbH
⁶⁾ Stearyl Alcohol: Stearyl alcohol, Merck KGaA
⁷⁾ Citric Acid: Citric acid, Sigma
⁸⁾ Tetrasodium EDTA: EDETA ® B powder, BASF Corporation
⁹⁾ Phenoxyethanol, Ethylhexylglycerin: Euxyl PE 9010, Schülke & Mayr

Damaged hairs treated with the rinse-off conditioners from example A1-b and A2-b are according to the panel test softer than damaged, untreated hairs.

### EXAMPLE A4

### Cosmetic Composition: Nourishing Styling Spray

The following example represents an inventive cosmetic composition A4 comprising the emulsion B1-a.

For the preparation of the cosmetic composition A4, 9.3 g of emulsion B1-a is diluted with 90.7 g of water. The active content of organopolysiloxanes in the cosmetic composition is 3.0%.

### COMPARATIVE EXAMPLE VA4

### Cosmetic Composition: Nourishing Styling Spray

The comparative experiment VA4 represents a noninventive cosmetic composition comprising aqueous dispersion V2 of a precrosslinked organopolysiloxane. For the preparation of the cosmetic composition of comparative experiment VA4, the procedure for example A4 was repeated, with the modification that the precrosslinked, noninventive emulsion V2 is used. 8.7 g of emulsion V2 was diluted with 91.3 g of water. The active content of organopolysiloxanes in the noninventive cosmetic composition VA4 is 3.0%.

Comparison of the Nourishing Styling Sprays of Inventive Example A4 with the Comparative Experiment VA4

To investigate the shaping properties of the nourishing styling sprays on hair, curls were produced as in the section “Curl retention at high humidity”, sprayed with 0.2 g of the respective spray, and curl retention was determined.

TABLE 13
Nourishing styling spray/Results of curl
retention at high humidity after 7 h.

| | Curl |
|---|---|
| | retention |
| Ex./Comp. | [%] |
| Ex. A4 | 73 |
| Comparative experiment VA4 | 30 |

The result was that curled locks of hair treated with the inventive styling spray from example A4 showed curl retention of 73% after 7 h. The curled locks of hair felt pleasantly soft and retained their shape even after combing. Combing the curled locks of hair was possible without resistance. For curled locks of hair treated with the spray from the noninventive comparative example VA4, curl retention of 30% was determined. The curl retention at high humidity is therefore significantly poorer for these curled locks of hair than for curled locks of hair treated with spray from the inventive example A4.

The invention claimed is:

1-15. (canceled)

16. An aqueous dispersion comprising precrosslinked organopolysiloxanes that contain on average at least one structural unit of the general formula
SiRO_{2/2}—Y—SiRO_{2/2} (I)
and units of the formula
R₂SiO_{2/2} (II)
where
Y denotes a radical of the formula
—R²—[NR³—R⁴]ₓ—NR³—OC—[C(Z¹)(H)]ₖ₁—[C(Z²)(H)]ₖ₂—CO—NR³—[R⁴—NR³]ₐ—R²—R₂SiO_{1/2}—[R₂SiO_{2/2}]_{b}—R₂SiO_{1/2}—R²—[NR³—R⁴]ₐ—NR³—OC—[C(Z²)(H)]ₖ₂—[C(Z¹)(H)]ₖ₁—CO—NR³—[R⁴—NR³]ₓ—R²—
R may be identical or different and denotes a monovalent SiC-bonded hydrocarbon radical that has 1 to 18 carbon atoms and may contain one or more heteroatoms from the group of N, P, S, O, and halogen,
R² may be identical or different and denotes a SiC-bonded, divalent linear or branched hydrocarbon radical having 3 to 18 carbon atoms,
R³ may be identical or different and denotes a hydrogen atom, an alkyl radical having 1 to 8 carbon atoms or an acyl radical, such as acetyl radical,
R⁴ may be identical or different and denotes a divalent hydrocarbon radical having 1 to 6 carbon atoms,
k1 is 0, 1, 2 or 3,
k2 is 0, 1, 2 or 3,
x is 0, 1, 2, 3 or 4,
a is 0, 1, 2, 3 or 4,
b is 0 or an integer from 1 to 500,
Z¹ denotes —OH, H or —NHR³,
Z² denotes —OH, —H or —NHR³.

17. The aqueous dispersion as claimed in claim 16, wherein the precrosslinked organopolysiloxanes contain siloxane units of the formula
R_{3-d}(OR¹)_{d}SiO_{1/2} (III)
where
R is as defined in claim 1,
R¹ may be identical or different and denotes a hydrogen atom or an alkyl radical that has 1 to 18 carbon atoms and may be interrupted by one or more separate oxygen atoms, and
d is 0 or 1.

18. The aqueous dispersion as claimed in claim 16, characterized in that Y a radical of the formula
—R²—[NH—CH₂CH₂]ₓ—NH—OC—CO—NH—R²—R₂SiO_{1/2}—[R₂SiO_{2/2}]_{b}—R₂SiO_{1/2}—R²—NH—OC—CO—NH—[CH₂CH₂—NH—]ₓ—R²—
where
R is as defined in claim 1,
x is 0 or 1,
b is 0 or an integer from 1 to 500, and
R² is a radical of the formula —(CH₂)₃— or —CH₂—CH(CH₃)—CH₂—.

19. A process for producing aqueous dispersions of precrosslinked organopolysiloxanes, wherein aqueous dispersions of aminoorganopolysiloxanes (1) of the formula
(R¹O)_{d}AₑR_{3-d-e}SiO(SiARO)ₚ(SiR₂O)_{q}SR_{3-d-e}Aₑ(OR¹)_{d} (IV)
where
A is an amino radical of the general formula
—R₂—[NR³—R⁴—]ₓNR³₂
R may be identical or different and denotes a monovalent SiC-bonded hydrocarbon radical that has 1 to 18 carbon atoms and may contain one or more heteroatoms from the group of N, P, S, O, and halogen,
R¹ may be identical or different and denotes a hydrogen atom or an alkyl radical that has 1 to 18 carbon atoms and may be interrupted by one or more separate oxygen atoms,
R² may be identical or different and denotes a SiC-bonded, divalent linear or branched hydrocarbon radical having 3 to 18 carbon atoms,
R³ may be identical or different and denotes a hydrogen atom, an alkyl radical having 1 to 8 carbon atoms or an acyl radical,
R⁴ may be identical or different and denotes a divalent hydrocarbon radical having 1 to 6 carbon atoms,
d is 0 or 1,
e is 0 or 1,
p is an integer that is at least 1 and at most 1000,
q is 0 or an integer from 1 to 2000, and
x is 0, 1, 2, 3 or 4,
are reacted with reactive esters (2) of the formula
R⁵—O₂C—[C(Z¹)(H)]ₖ₁—[C(Z²)(H)]ₖ₂—CO—NR³—[R⁴—NR³]ₐ—R²—R₂SiO_{1/2}—[R₂SiO_{2/2}]_{b}—R₂SiO_{1/2}—R²[NR³—R⁴]ₐ—NR³—OC—[C(Z²)(H)]ₖ₂—[C(Z¹)(H)]ₖ₁—CO₂—R⁵ (V)
where R, R², R³, and R⁴ are as defined above,
R⁵ may be identical or different and denotes an O-bonded, saturated or unsaturated, linear or branched, monovalent hydrocarbon radical that has 1-20 carbon atoms per radical and may contain one or more heteroatoms from the group of N, P, S, O and halogen,
k1 is 0, 1, 2 or 3,
k2 is 0, 1, 2 or 3,
a is 0, 1, 2, 3 or 4,
b is 0 or an integer from 1 to 500,
Z¹ denotes —OH, —H or —NHR³, and
Z² denotes —OH, —H or —NHR³.

20. The process as claimed in claim 19, wherein A is an amino radical of the formula
—R²—[NH—CH₂CH₂—]ₓNH₂
where
x is 0 or 1 and
R² is a radical of the formula —(CH₂)₃— or —CH₂—CH(CH₃)—CH₂—.

21. The process as claimed in claim 19, wherein the reactive esters (2) are oxalamido-methyl-ester-terminated organopolysiloxanes and oxalamido-ethyl-ester-terminated organopolysiloxanes.

22. A precrosslinked organopolysiloxane that contains on average at least one structural unit of the general formula
SiRO_{2/2}—Y—SiRO_{2/2} (I)
and units of the formula
R₂SiO_{2/2} (II)
where
Y denotes a radical of the formula
—R²—[NR³—R⁴]ₓ—NR³—OC—[C(Z¹)(H)]ₖ₁—[C(Z²)(H)]ₖ₂—CO—NR³—[R⁴—NR³]ₐ—R²—R₂SiO_{1/2}—[R₂SiO_{2/2}]_{b}—R₂SiO_{1/2}—R²—[NR³—R⁴]ₐ—NR³—OC—[C(Z²)(H)]ₖ₂—[C(Z¹)(H)]ₖ₁—CO—NR³—[R⁴—NR³]ₓR²
R, R², R³, R⁴, k1, k2, x, a, b, Z¹, and Z² are as defined in claim 16.

23. The precrosslinked organopolysiloxane as claimed in claim 22, further containing siloxane units of the formula
R_{3-d}(OR¹)_{d}SiO_{1/2} (III)
where
R is as defined in claim 1 and
d and R¹ are as defined in claim 2.

24. A process for producing precrosslinked organopolysiloxanes, wherein aminoorganopolysiloxanes of the formula
(R¹O)_{d}AₑR_{3-d-e}SiO(SiARO)ₚ(SiR₂O)_{q}SiR_{3-d-e}Aₑ(OR¹)_{d} (IV)
are reacted with reactive esters of the formula
R⁵—O₂C—[C(Z¹)(H)]ₖ₁—[C(Z²)(H)]ₖ₂—CO—NR³—[R⁴—NR³]ₐ—R²—R₂SiO_{1/2}—[R₂SiO_{2/2}]_{b}—R₂SiO_{1/2}—R²[NR³—R⁴]ₐ—NR³—OC—[C(Z²)(H)]ₖ₂—[C(Z¹)(H)]ₖ₁—CO₂—R⁵ (V)
where
A, R, R¹, R², R³, R⁴, R⁵, d, e, p, q, k1, k2, a, b, Z¹, and Z² are as defined in claim 19,
and the resulting precrosslinked organopolysiloxanes are optionally subsequently emulsified in water.

25. A cosmetic composition comprising aqueous dispersions of precrosslinked organopolysiloxanes as claimed in claim 16.

26. The cosmetic composition as claimed in claim 25, further comprising conditioning agents.

27. The use of the cosmetic composition as claimed in claim 25 for treating keratin fibers.

28. The use of the cosmetic composition as claimed in claim 25 for conditioning keratin fibers.

29. The use as claimed in claim 27, wherein the keratin fibers are hair.

30. The use as claimed in claim 28, wherein the keratin fibers are hair.

31. A process for treating keratin-containing fibers by applying the cosmetic composition of claim 25 to the keratin fibers and then optionally rinsing with water.
